(19) United States
(12) Patent Application Publication
(10) Pub. No.: US 2014/0343009 A1
(43) Pub. Date: Nov. 20, 2014

(54) METHODS FOR TREATING METHYLMALONIC ACIDEMIA

(71) Applicant: PTC Therapeutics Inc., South Plainfield, NJ (US)

(72) Inventor: Jay Allan Barth, Teaneck, NJ (US)

(73) Assignee: PTC Therapeutics Inc., South Plainfield, NJ (US)

(21) Appl. No.: 14/447,735

(22) Filed: Jul. 31, 2014

Related U.S. Application Data
(63) Continuation of application No. 13/514,324, filed on Aug. 7, 2012.
(60) Provisional application No. 61/285,934, filed on Dec. 11, 2009.

(51) Int. Cl. C07D 271/06; A61K 31/714; A61K 31/4164; A61K 31/4245; A61K 31/205

(52) U.S. Cl. CPC C07D 271/06; A61K 31/4245; A61K 31/205; A61K 31/4164; A61K 31/714
USPC 514/52; 514/364

(57) ABSTRACT

Methods for treating methylmalonic acidemia in which at least one allele of a gene associated with MMA (e.g., the MUT, MMAA, or MMAB gene) contains a mutation (e.g., nonsense mutation) that results in a premature stop codon in RNA encoded by an allele of the gene associated with MMA involving the administration of a compound that promotes readthrough of RNA (e.g., messenger RNA) containing a premature stop codon encoded by an allele of the gene associated with MMA are described. The compound can be administered as a single-agent therapy or in combination with one or more additional therapies to a human in need of such treatment.

This application is a continuation of U.S. patent application Ser. No. 13/514,324, filed Aug. 7, 2012, which is a U.S. national stage application of International Patent Application No. PCT/US2010/060048, filed Dec. 13, 2010, which claims the benefit of and priority to U.S. provisional application No. 61/285,934, filed Dec. 11, 2009, the content of each of which is incorporated by reference herein in its entirety.

### 1. INTRODUCTION

Methods for treating methylmalonic acidemia associated with a mutation (e.g., nonsense mutation) involving the administration of a compound that promotes readthrough of RNA containing a premature stop codon are described. The compound can be administered as a single-agent therapy or in combination with one or more additional therapies to a human in need of such treatment.

### 2. BACKGROUND

Methylmalonic acidemia (also known as methylmalonic aciduria) is a rare autosomal recessive genetic disorder caused by deficiencies of the enzyme methylmalonyl-Coenzyme A (CoA) mutase (MCM) or by defects in the synthesis of adenosylcobalamin (AdoCbl), the cofactor of MCM (Fowler et al., 2008, J. Inherit. Metab. Dis. 31: 350-360). MCM plays a key role in the final catabolic pathway of the branched chain amino acids isoleucine, valine, methionine and threonine, as well as erythrocyte odd long-chain fatty acids (OLCFAs) and the side chains of cholesterol (Horster et al., 2007, Pediatr. Res. 62: 225-230). MCM catalyzes the conversion of methylmalonyl-CoA to succinyl-CoA, which enters the tricarboxylic acid cycle. Deficiency of MCM or AdoCbl leads to accumulation of methylmalonic acid (MMacid) in body fluids and in urine (Zwickler et al., 2008, J. Inherit. Metab. Dis. 31: 361-367). MMacid and its metabolites, propionyl-CoA and 2-methylcitrate, are believed to act synergistically to inhibit mitochondrial energy metabolism (the tricarboxylic acid cycle and the mitochondrial respiratory chain) (Horster et al., 2004, Pediatr. Nephrol. 19: 1071-1074; Morath et al., 2008, J. Inherit. Metab. Dis. 31: 35-43). These errors result in the various clinical manifestations of the disease.

The prevalence of MMA is not known precisely, but is thought to occur in about 1 in 48,000 to 1 in 250,000 individuals (Lempp et al., 2007, Mol. Genet. Metab. 90: 284-290). There are 4 types of isolated MMA, defined based on the enzymatic defect: mut⁰ and mut⁻, which represent complete and partial deficiency of MCM, respectively; and cblA and cblB, which represent defects in AdoCbl synthesis. Mut⁰ is the most common form of MMA, occurring in about 40 to 45% of patients; mut⁻ occurs in about 20% of patients; and about 30 to 40% of patients have cblA or cblB, with cblA more frequent than cblB (Zwickler et al., 2008, J. Inherit Metab. Dis. 31: 361-367; Merinero et al., 2008, J. Inherit. Metab. Dis. 31: 55-66). Consistent with the general degree of enzymatic deficiency, the most severe form of MMA is mut⁰, followed by cblB, with mut⁻ and cblA being the less severe forms (Horster et al., 2007, Pediatr. Res. 62: 225-230).

Patients are also characterized as being responsive or non-responsive to cobalamin (vitamin B12). There is no universally accepted definition of a response. A standardized definition for cobalamin responsiveness of a >50% decrease in urine or plasma MMacid levels with administration of cobalamin has been proposed (Fowler et al., 2008, J. Inherit. Metab. Dis. 31: 350-360); however, a range of 30 to 90% reduction in urinary MMacid levels with administration of cobalamin is used to define cobalamin responsiveness across different centers in Europe (Zwickler et al., 2008, J. Inherit. Metab. Dis. 31: 361-367). Cobalamin responsiveness occurs rarely, if ever, in mut⁰ patients; occasionally in cblB patients; often in mut⁻ patients; and in nearly all cblA patients (Horster et al., 2007, Pediatr. Res. 62: 225-230; Fowler et al., 2008, J. Inherit. Metab. Dis. 31: 350-360).

The age of onset and clinical manifestations depend on the type of MMA, and are related to the severity of the enzymatic defect. The median age of onset is 5 days in mut⁰, 10 days in cblB, 25 days in cblA, and 75 days in mut⁻ (Horster et al., 2007, Pediatr. Res. 62: 225-230). The disease generally has a neonatal onset in the more severe types, with metabolic acidosis, lethargy, vomiting, dehydration, and hypotonia. If not managed successfully, these events can lead to coma and even death (Horster et al., 2004, Pediatr. Nephrol. 19: 1071-1074). Patients with neonatal onset who survive beyond infancy, and patients with late onset (i.e., beyond the neonatal period), have variable clinical manifestations and outcomes. Episodes of metabolic decompensation are often precipitated by febrile infectious illnesses and are characterized by vomiting, hypotonia, and alteration of consciousness associated with metabolic acidosis and hyperammonemia. These metabolic crises tend to become less frequent with age (Horster et al., 2004, Pediatr. Nephrol. 19: 1071-1074; Touati et al., 2006, J. Inherit. Metab. Dis. 29: 288-298).

Neurologic manifestations occur in 50 to 80% of patients, more commonly in patients with mut⁰, and include movement disorders, seizures, mental retardation, and cognitive dysfunction (Horster et al., 2007, Pediatr. Res. 62: 225-230; Cosson et al., 2009, Mol. Genet. Metab. 97: 172-178). Neurologic abnormalities are thought to be associated primarily with acute metabolic decompensations and related acidosis, hyperammonemia, dehydration, shock, and apnea (Nyhan et al., 2002, Eur. J. Pediatr. 161: 377-379). Metabolic stroke, affecting primarily the basal ganglia, may occur (Cosson et al., 2009, Mol. Genet. Metab. 97: 172-178). Central nervous system trapping of MMacid, propionyl-CoA, and 2 methylcitrate is considered to be the basis for chronic neurologic complications (Morath et al., 2008, J. Inherit. Metab. Dis. 31: 35-43).

Chronic progressive renal failure occurs in about 60 to 65% of mut⁰ and cblB patients, and in up to 20% of mut⁻ and cblA patients (Horster et al., 2007, Pediatr. Res. 62: 225-230). Preclinical and clinical data support the concept that MMacid is a nephrotoxin (Morath et al., 2008, J. Inherit. Metab. Dis. 31: 35-43). The renal disease is a tubulointerstitial nephritis with mononuclear cell infiltration, interstitial fibrosis, and tubular atrophy, and is reflected by reduction in glomerular filtration rate (GFR) and progressively increasing serum creatinine levels (Schmitt et al., 2004, Ped. Nephrol. 19: 1182-1184; Horster et al., 2004, Pediatr. Nephrol. 19: 1071-1074). It is difficult to predict the clinical course of renal disease in individual patients, although renal insufficiency occurs earlier and is more severe in the more severe types of MMA (Cosson et al., 2009, Mol. Genet. Metab. 97: 172-178; Horster et al., 2007, Pediatr. Res. 62: 225-230). Other potential complications of MMA include anorexia, vomiting, failure to thrive, and pancreatitis (Horster et al., 2007, Pediatr. Res. 62: 225-230; Cosson et al., 2009, Mol. Genet. Metab. 97: 172-178).

The preliminary diagnosis of MMA is considered in patients presenting with clinical signs and symptoms consistent with MMA and elevated MMacid levels. The normal urinary MMacid level is <4 mmol/mol creatinine (Venditti, 2007, Gene Reviews). In general, the more severe types, mut⁰ and cblB, have a have higher urinary MMacid levels (about 5,000 to >10.000 mmol/mol creatinine) compared to the less severe types, mut⁻ and cblA, that have lower urinary MMacid levels (<1,000 to >5.000 mmol/mol creatinine) (Horster et al., 2007, Pediatr. Res. 62: 225-230; Fowler et al., 2008, J. Inherit. Metab. Dis. 31: 350-360). Plasma MMacid is measured less frequently than urinary MMacid in clinical practice (Zwickler et al., 2008, J. Inherit. Metab. Dis. 31: 361-367); a normal plasma MMacid level is <0.27 μmol/L (Fowler et al., 2008, J. Inherit. Metab. Dis. 31: 350-360).

A definite diagnosis of MMA is based on enzyme studies in cultured fibroblasts obtained from skin biopsies. The ¹⁴C-propionate incorporation assay measures the overall conversion of propionate to succinate, and assay of MCM activity distinguishes mut from cblA/cblB. Mut⁰ is defined as very low MCM activity with <1.5-fold increase in propionate incorporation after administration of hydroxyl-cobalamin (OH-Cbl), whereas mut− is defined as low to moderate residual mutase activity with at least 1.5-fold increase in propionate incorporation after administration of OH-Cbl (Fowler et al., 2008, J. Inherit. Metab. Dis. 31: 350-360; Horster et al., 2007, Pediatr. Res. 62: 225-230).

Mutation analysis is also used to determine the enzymatic defect in MMA. The MUT gene, for MCM, maps to 6p21, contains 13 exons, and codes for a 750-amino-acid protein. Approximately 200 mutations have been identified in this gene (Fowler et al., 2008, J. Inherit. Metab. Dis. 31: 350-360). The gene for cblA, MMAA, is located at 4q31.1-q31.2, consists of 7 exons, and codes for a 418-amino-acid protein. At least 28 mutations have been identified in cblA patients (Fowler et al., 2008, J. Inherit. Metab. Dis. 31: 350-360). The gene for cblB, MMAB, maps to 12q24, contains 9 exons, and codes for a 250-amino-acid protein. At least 24 mutations have been identified in the MMAB gene (Fowler et al., 2008, J. Inherit. Metab. Dis. 31: 350-360). Mutations (e.g., nonsense mutations) in the MUT gene encoding the protein, that result in a premature stop codon in RNA encoded by the MUT gene, are the basis for MMA in approximately 5 to 20% of patients with such mutations in the MUT gene, and approximately 20 to >50% of patients with mutations (e.g., nonsense mutations) in the AdoCbl genes, cblB and cblA genes encoding the protein, respectively, result in a premature stop codon in RNA encoded by the AdoCbl genes, cblB and cblA genes (Acquaviva et al., 2005, Human Mutation 25: 167-176; Cosson et ah, 2009, Mol. Genet. Metab. 97: 172-178; Lempp et al., 2007, Mol. Genet. Metab. 90: 284-290; Lerner-Ellis et al., 2006, Mol. Genet. Metab. 87: 219-225; Merinero et al., 2008, J. Inherit. Metab. Dis. 31: 55-66; Martinez et al., 2005, Mol. Genet. Metab. 84: 317-325).

In cobalamin-responsive patients, supplementation is usually given in the form of oral or intramuscular hydroxycobalamin (Zwickler et al., 2008, J. Inherit. Metab. Dis. 31: 361-367). Other than cobalamin, there is no specific medical treatment for MMA. The disease is commonly managed with a low protein diet, based on patient weight and age, and in some cases supplemented with an amino acid mixture that is deficient in branched-chain amino acids (de Baulny et al., 2005, J. Inherit. Metab. Dis. 28: 415-423; Horster et al., 2004, Pediatr. Nephrol. 19: 1071-1074; Zwickler et al., 2008, J. Inherit. Metab. Dis. 31: 361-367). Feeding problems and growth retardation are frequent, so patients often receive enteral nutrition (de Baulny et al., 2005, J. Inherit. Metab. Dis. 28: 415-423). Patients with MMA are often treated with L-carnitine, because they tend to have low levels of total and free carnitine (Horster et al., 2004, Pediatr. Nephrol. 19: 1071-1074). Antibiotics, such as metronidazole, may be given to reduce gut flora production of propionate, which is an additional source of MMacid (Zwickler et al., 2008, J. Inherit. Metab. Dis. 31: 361-367).

Liver, kidney, or combined liver-kidney transplantation has been employed in MMA, although the benefit is not clearly established and transplantation has been associated with significant mortality (de Baulny et al., 2005, J. Inherit. Metab. Dis. 28: 415-423; Kaplan et al., 2006, Mol. Genet. Metab. 88: 322-326). Renal transplantation may be performed in patients with end-stage renal disease to restore kidney function and replace the deficient enzyme (Lubrano et al., 2007, Pediatr. Nephrol. 22: 1209-1214). Liver or combined liver-kidney transplantation has been performed in some centers to provide enzyme replacement to improve metabolic control. MMacid levels may decrease by >80% following combined liver kidney transplantation. Such reductions can prevent further episodes of metabolic decompensation (Kasahara et al., 2006, Pediatr. Transplantation 10: 943-947; McGuire et al., 2008, Mol. Genet. Metab. 93: 22-29). However, organ transplantation does not prevent progression of neurologic complications, and liver transplantation does not prevent progression of renal disease (Chakrapani et al., 2002, J. Pediatr. 140: 261-263; de Baulny et al., 2005, J. Inherit. Metab. Dis. 28: 415-423; Kaplan et al., 2006, Mol. Genet. Metab. 88: 322-326; McGuire et al., 2008, Mol. Genet. Metab. 93: 22-29; Nyhan et al., 2002, Eur. J. Pediatr. 161: 377-379).

Mortality in MMA is generally related to the severity of the enzymatic subgroup, being highest in mut⁰ patients. Mortality has fallen over time with advances in clinical management of the disorder (de Baulny et al., 2005, J. Inherit. Metab. Dis. 28: 415-423; Horster et al., 2007, J. Pediatr. Res. 62: 225-230). In an earlier series of patients, mortality was reported as ≧80% in patients with neonatal onset and as high as 40% in patients with late onset disease (van de Meer et al., 1994, J. Pediatr. 25: 903-908; Horster et al., 2007, J. Pediatr. Res. 62: 225-230). In more recent reports, disease-related mortality was 20% for all types of MMA, including neonatal and late onset forms (de Baulny et al., 2005, J. Inherit. Metab. Dis. 28: 415-423). Death is usually associated with metabolic decompensation episodes in younger patients and, in addition, is also associated with renal failure or organ transplantation in older patients (de Baulny et al., 2005, J. Inherit. Metab. Dis. 28: 415-423; Touati et al., 2006, J. Inherit. Metab. Dis. 29: 288-298).

Collectively, these data support the development of a new therapy that might safely and conveniently overcome the underlying cause of MMA. An oral treatment that could persistently reduce MMacid levels in peripheral and central nervous system tissues could potentially reduce the frequency of metabolic crises, limit the progression of neurological and renal dysfunction, allow easing of dietary restrictions, and ultimately improve survival.

### 3. SUMMARY

Methods for treating methylmalonic acidemia (hereinafter “MMA”), e.g., mut⁰ MMA, mut⁻ MMA, cblA MMA, or cblB MMA, associated with the presence of a mutation (e.g., nonsense mutation) in the gene encoding the protein, that result in a premature stop codon in RNA encoded by the gene, are described involving the administration of compounds having the formulas set forth herein (“Compound”) to a human subject in need of such treatment. In certain embodiments, provided herein are Compounds for use in the treatment of MMA.

The Compound can be administered as a single-agent therapy to a human need of such treatment. Alternatively, the Compound can be administered in combination with one or more additional therapies to a human in need of such treatment.

Without being bound by any theory, the therapies described herein are based, in part, on the ability of a Compound to promote readthrough of RNA containing a premature stop codon. In particular, the therapies described herein are based, in part, on the ability of a Compound to selectively promote readthrough of RNA (e.g., mRNA) containing a premature stop codon without promoting readthrough of normal stop codons.

### 3.1 Definitions

As used herein, the term “about” means an acceptable error for a particular value as determined by one of ordinary skill in the art, which depends in part on how the value is measured or determined. In certain embodiments, the term “about” means within 1, 2, 3, or 4 standard deviations. In certain embodiments, the term “about” means within 50%, 20%, 15%, 10%, 9%, 8%, 7%, 6%, 5%, 4%, 3%, 2%, 1%, 0.5%, or 0.05% of a given value or range. In certain embodiments, it is contemplated that the values preceded by the term “about” are exact.

As used herein, the term “Compound” includes compounds of formula I, II and III as well as individual compounds provided herein, and pharmaceutically acceptable salts, hydrates, solvates and stereoisomers, including enantiomers, diastereomers, racemates and mixtures of stereoisomers, thereof. In specific embodiments, a Compound promotes readthrough of RNA containing one or more premature stop codons, enabling translation of a functional protein. In certain specific embodiments, a Compound does not promote readthrough of normal stop codons, alter RNA (e.g., mRNA) levels, or affect the process of mRNA decay. In certain specific embodiments, a Compound prevents premature termination of protein synthesis. In one embodiment, an individual Compound is 3-[5-(2-Fluoro-phenyl)-[1,2,4]oxadiazol-3-yl]-benzoic acid or a pharmaceutically acceptable salt, hydrate or solvate thereof.

As used herein, the term “dose(s)” means a quantity of a Compound to be administered at one time.

As used herein, the terms “dosing regimen” and “dosage(s)” mean the amount of a Compound given per time unit and the duration of administration.

As used herein, “premature translation termination” refers to the result of a mutation that changes a codon corresponding to an amino acid to a stop codon.

As used herein, a “premature termination codon,” a “premature stop codon” or a “premature stop mutation” refers to the occurrence of a stop codon where a codon corresponding to an amino acid should be. Such an occurrence is the result of one or more nucleotide changes in an exon or intron that cause disease by premature termination of RNA (e.g., mRNA) translation before a functional protein is generated. In certain embodiments, such nucleotide changes include a frameshift, insertion, deletion, point, substitution, transition, transversion, or chain termination mutation. In certain embodiments, the mutation is one or more point mutations. In a specific embodiment, the mutation is a nonsense mutation. In another specific embodiment, the mutation is not a nonsense mutation. In certain other embodiments, the mutation is the result of defective DNA or RNA editing. In certain other embodiments, the mutation is the result of defective or alternative splicing.

As used herein, a “nonsense mutation” is a mutation in DNA that, when transcribed to RNA, results in a codon that is interpreted as a stop codon by the ribosomal cellular translational machinery. In other words, a nonsense mutation is a mutation that results in the occurrence of a stop codon where a codon corresponding to an amino acid should be (i.e., a premature stop codon).

As used herein, the term “effective amount” in the context of administering a Compound to a subject refers to the amount of a Compound that results in a beneficial or therapeutic effect. In specific embodiments, an “effective amount” of a Compound refers to an amount of a Compound which is sufficient to achieve at least one, two, three, four or more of the following effects: (i) the reduction or amelioration of the severity of one or more MMA symptoms; (ii) the reduction in the duration of one or more symptoms associated with MMA; (iii) the prevention in the recurrence of a symptom associated with MMA; (iv) the reduction in hospitalization of a subject; (v) a reduction in hospitalization length; (vi) the increase in the survival of a subject; (vii) the enhancement or improvement of the therapeutic effect of another therapy; (viii) an improvement in developmental or cognitive ability; (ix) a decrease in the frequency and/or number of metabolic decompensation episodes; (x) an improvement in control of muscle contraction; (xi) a reduction in mortality; (xii) an increase in the survival rate of patients; (xiii) a decrease in hospitalization rate; (xiv) the prevention of the development or onset of one or more symptoms associated with MMA; (xv) the reduction in the number of symptoms associated with MMA; (xvi) an decrease in the concentration of MMacid in biological fluids (e.g., plasma or urine); (xvii) a decrease in the concentration of metabolites of MMacid, such as propionylcarnitine or methylcitrate, in biological fluids (e.g., plasma or urine); (xviii) a decrease in erythrocyte OLCFA levels; (xix) an increase in the urinary urea:MMacid ratio; (xx) an increase in symptom-free survival of MMA patients; (xxi) an improvement in renal function; and (xxii) improvement in quality of life as assessed by methods well known in the art. In specific embodiments, an “effective amount” of a Compound refers to an amount of a Compound specified herein, e.g., in Section 4.4, infra.

As used herein, the term “elderly human” refers to a human adult 65 years or older.

As used herein, the term “functional” in the context of a functional readthrough protein refers to the amount of a protein that has enough of the activity or functions of the corresponding wild-type protein to have a beneficial effect in a cell or subject which does not endogenously produce or produces insufficient amounts of the wild-type protein as the result of a mutation (e.g., nonsense mutation) in the gene encoding the protein, which mutation results in a premature stop codon in RNA encoded by the gene.

In a specific embodiment, a functional readthrough protein produced from a MUT gene comprising a mutation (e.g., nonsense mutation) that results in a premature stop codon in RNA encoded by the MUT gene is able to catalyze the conversion of L-methylmalonyl-CoA to succinyl-CoA. In certain embodiments, a functional readthrough protein produced from a MUT gene comprising a mutation (e.g., nonsense mutation) that results in a premature stop codon in RNA encoded by the MUT gene is able to catalyze the conversion of at least 0.1%, 0.5%, 1%, 2%, 2.5%, 5%, 10%, 20%, 25%, 30%, 35% 40%, 45%, 50%, 55%, 60%, 65%, 70%, 75%, 80%, 85% or more of L-methylmalonyl-CoA to succinyl-CoA. In some embodiments, a functional readthrough protein produced from a MUT gene comprising a mutation (e.g., nonsense mutation) that results in a premature stop codon in RNA encoded by the MUT gene is able to catalyze the conversion of a range of from 0.1% to 5%, 5% to 25%, 10% to 25%, 10% to 30%, 20% to 40%, 10% to 50%, 20% to 50%, 10% to 75%, 20% to 75%, 30% to 75%, 30% to 85%, or 25% to 100% of L-methylmalonyl-CoA to succinyl-CoA.

In a specific embodiment, a functional readthrough protein produced from a MMAA or MMAB gene comprising a mutation (e.g., nonsense mutation) that results in a premature stop codon in RNA encoded by the MMAA or MMAB gene achieves enzymatic activity sufficient for the synthesis of AdoCbl. In certain embodiments, a functional readthrough protein produced from a MMAA or MMAB gene comprising a mutation (e.g., nonsense mutation) that results in a premature stop codon in RNA encoded by the MMAA or MMAB gene achieves at least 0.1%, 0.5%, 1%, 2%, 2.5%, 5%, 10%, 20%, 25%, 30%, 35% 40%, 45%, 50%, 55%, 60%, 65%, 70%, 75%, 80%, 85% or more of the enzymatic activity sufficient for the synthesis of AdoCbl. In some embodiments, a functional readthrough protein produced from a MMAA or MMAB gene comprising a mutation (e.g., nonsense mutation) that results in a premature stop codon in RNA encoded by the MMAA or MMAB gene achieves a range of from 0.1% to 5%, 5% to 25%, 10% to 25%, 10% to 30%, 20% to 40%, 10% to 50%, 20% to 50%, 10% to 75%, 20% to 75%, 30% to 75%, 30% to 85%, or 25% to 100% of the enzymatic activity sufficient for the synthesis of AdoCbl.

In another specific embodiment, a functional readthrough protein produced from a MMAA or MMAB gene comprising a mutation (e.g., nonsense mutation) that results in a premature stop codon in RNA encoded by the MMAA or MMAB gene achieves sufficient enzymatic activity to synthesize at least 0.1%, 0.5%, 1%, 2%, 2.5%, 5%, 10%, 20%, 25%, 30%, 35% 40%, 45%, 50%, 55%, 60%, 65%, 70%, 75%, 80%, 85% or more of the active form of AdoCbl found in healthy individuals. In some embodiments, a functional readthrough protein produced from a MMAA or MMAB gene comprising a mutation (e.g., nonsense mutation) that results in a premature stop codon in RNA encoded by the MMAA or MMAB gene achieves sufficient enzymatic activity to synthesize a range of from 0.1% to 5%, 5% to 25%, 10% to 25%, 10% to 30%, 20% to 40%, 10% to 50%, 20% to 50%, 10% to 75%, 20% to 75%, 30% to 75%, 30% to 85%, or 25% to 100% of the active form of AdoCbl found in healthy individuals.

As used herein, the term “functional readthrough protein” refers to a functional protein produced as a result of readthrough of a premature stop codon in a RNA (e.g., mRNA) transcribed from a gene. In a specific embodiment, the term “functional readthrough protein” refers to a functional protein produced as a result of readthrough of a premature stop codon in a RNA transcribed from a gene comprising one or more nucleotide changes in an exon or intron that result in the occurrence of a stop codon where a codon corresponding to an amino acid should be. In certain embodiments, such nucleotide changes include a frameshift, insertion, deletion, point, substitution, transition, transversion, or chain termination mutation. In certain embodiments, the mutation is one or more point mutations. In a specific embodiment, the mutation is a nonsense mutation. In certain embodiments, the functional readthrough protein is composed of the same amino acid as the corresponding wild-type protein encoded by a gene without the mutation. In other embodiments, the functional readthrough protein is a functional non-wild-type protein.

As used herein, the term “human adult” refers to a human that is 18 years or older.

As used herein, the term “human child” refers to a human that is 1 year to 18 years old.

As used herein, the term “human infant” refers to a human that is a newborn to 1 year old.

As used herein, the term “human toddler” refers to a human that is 1 year to 3 years old.

As used herein, the term “non-wild-type protein” refers to a protein having an amino acid sequence that is different from the corresponding wild-type protein. In certain embodiments, the non-wild-type protein only differs from the corresponding wild-type protein at the amino acid residue(s) in the non-wild-type protein that was inserted at the position(s) encoded by a premature stop codon(s). In other embodiments, the non-wild-type protein differs from the corresponding wild-type protein: (i) at an amino acid residue(s) in the non-wild-type protein(s) that was inserted at the position encoded by a premature stop codon(s); and (ii) at an amino acid residue(s) in the non-wild-type protein other than those encoded by a premature stop codon(s).

As used herein, the term “pharmaceutically acceptable salts” refer to salts prepared from pharmaceutically acceptable non-toxic acids or bases including inorganic acids and bases and organic acids and bases. Suitable pharmaceutically acceptable base addition salts for the compound of the present invention include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Other examples of salts are well known in the art, see, e.g., Remington's Pharmaceutical Sciences, 18th ed., Mack Publishing, Easton Pa. (1990).

As used herein, the term “subject” and “patient” are used interchangeably to refer to an animal (e.g., cow, horse, sheep, pig, chicken, turkey, quail, cat, dog, mouse, rat, rabbit, guinea pig, etc.), preferably a mammal such as a non-primate and a primate (e.g., monkey and human), most preferably a human. In a specific embodiment, a subject is an animal that has or is diagnosed with MMA, which is associated with a gene comprising a mutation (e.g., nonsense mutation) that results in a premature stop codon in RNA encoded by the gene associated with MMA. See Section 4.3, infra for more information concerning patients treated for MMA in accordance with the methods provided herein.

As used herein, the terms “therapies” and “therapy” can refer to any protocol(s), method(s), compositions, formulations, and/or agent(s) that can be used in the prevention, treatment, management, or amelioration of a condition or disorder or symptom thereof (e.g., MMA or a symptom or condition associated therewith). In certain embodiments, the terms “therapies” and “therapy” refer to drug therapy, adjuvant therapy, radiation, surgery, biological therapy, supportive therapy, and/or other therapies useful in treatment, management, prevention, or amelioration of a condition or disorder or a symptom thereof (e.g., MMA or a symptom or condition associated therewith). In certain embodiments, the term “therapy” refers to a therapy other than a Compound or a pharmaceutical composition thereof. In specific embodiments, an “additional therapy” and “additional therapies” refer to a therapy other than a treatment using a Compound or a pharmaceutical composition thereof. In a specific embodiment, a therapy includes the use of a Compound as an adjuvant therapy. For example, using a Compound in conjunction with a drug therapy, biological therapy, surgery, and/or supportive therapy.

As used herein, the term “wild-type” in the context of a protein refers to a protein that is found in nature (often (but not necessarily) it is the predominant protein) and is designated as a standard or reference protein.

As used herein, the term “unit dosage form(s)” includes tablets; caplets; capsules, such as soft elastic gelatin capsules; sachets; packets; cachets; troches; lozenges; dispersions; granules, powders; solutions; gels; liquid dosage forms suitable for oral or mucosal administration to a patient, including suspensions (e.g., aqueous or non-aqueous liquid suspensions), emulsions (e.g., oil-in-water emulsions, or a water-in-oil liquid emulsion), solutions, and elixirs; and sterile solids (e.g., crystalline or amorphous solids) that can be dissolved or suspended to provide liquid dosage forms suitable for oral or parenteral administration to a patient. The unit dosage form does not necessarily have to be administered as a single dose.

As used herein, unless otherwise specified, the term “substituted” means that a Compound is substituted at one or more positions by one or more substituents where allowed by available valences. Examples of radicals that may be used as substituents are known to those skilled in the art, including those of the compounds described herein.

As used herein, unless otherwise specified, the term “alkyl” means a saturated straight chain or branched non-cyclic hydrocarbon radical having from 1 to 20 carbon atoms, preferably 1-10 carbon atoms and most preferably 1-4 carbon atoms. Representative saturated straight chain (C₁-C₁₀)alkyls include -methyl, -ethyl, -n-propyl, -n-butyl, -n-pentyl, -n-hexyl, -n-heptyl, -n-octyl, -n-nonyl and -n-decyl; while saturated branched (C₁-C₁₀)alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted. Unsaturated alkyl radicals include alkenyl radicals and alkynyl radicals, which are discussed below.

As used herein, unless otherwise specified the term “alkenyl” means a straight chain or branched non-cyclic hydrocarbon radical having from 2 to 20 carbon atoms, more preferably 2-10 carbon atoms, most preferably 2-6 carbon atoms, and including at least one carbon-carbon double bond. Representative straight chain and branched (C₂-C₁₀)alkenyls include -vinyl, -allyl, -1-butenyl, -2-butenyl, -isobutylenyl, -1-pentenyl, -2-pentenyl, methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. The double bond of an alkenyl radical can be unconjugated or conjugated (where allowed by available valences) to another unsaturated moiety. An alkenyl radical can be unsubstituted or substituted.

As used herein, unless otherwise specified the term “alkynyl” means a straight chain or branched non-cyclic hydrocarbon radical having from 2 to 20 carbon atoms, more preferably 2-10 carbon atoms, most preferably 2-6 carbon atoms, and including at least one carbon-carbon triple bond. Representative straight chain and branched —(C₂-C₁₀)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. The triple bond of an alkynyl radical can be unconjugated or conjugated to another unsaturated moiety. An alkynyl radical can be unsubstituted or substituted.

As used herein, unless otherwise specified the term “halogen” or “halo” means fluorine, chlorine, bromine, or iodine.

As used herein, unless otherwise specified the term “alkylsulfonyl” means a radical of the formula: -alkyl-SO₃H or —SO₃-alkyl, wherein alkyl is defined as above, including —SO₂—CH₃, —SO₂—CH₂CH₃, —SO₂—(CH₂)₂CH₃, —SO₂—(CH₂)₃CH₃, —SO₂—(CH₂)₄—CH₃, —SO₂—(CH₂)₅CH₃, and the like.

As used herein, unless otherwise specified the term “carboxyl” and “carboxy” mean a radical of the formula: —COOH or —CO₂H.

As used herein, unless otherwise specified the term “alkoxy” means a radical of the formula: —O-(alkyl), wherein alkyl is defined above, including —OCH₃, —OCH₂CH₃, —O(CH₂)₂CH₃, —O(CH₂)₃CH₃, —O(CH₂)₄—CH₃, —O(CH₂)₅CH₃, and the like.

As used herein, unless otherwise specified the term “alkoxycarbonyl” means a radical of the formula: —C(═O)O-(alkyl), wherein alkyl is defined above, including —C(═O)O—CH₃, —C(═O)O—CH₂CH₃, —C(═O)O—(CH₂)₂CH₃, —C(═O)O—(CH₂)₃CH₃, —C(═O)O—(CH₂)₄—CH₃, —C(═O)O—(CH₂)₅CH₃, and the like. In a preferred embodiment, the esters are biohydrolyzable (i.e., the ester is hydrolyzed to a carboxylic acid in vitro or in vivo).

As used herein, unless otherwise specified the term “alkoxyalkyl” means a radical of the formula: -(alkyl)-O-(alkyl), wherein each “alkyl” is independently an alkyl group as defined above, including —CH₂OCH₃, —CH₂OCH₂CH₃, —(CH₂)₂OCH₂CH₃, —(CH₂)₂O(CH₂)₂CH₃, and the like.

As used herein, unless otherwise specified the term “aryl” means an aromatic carbocyclic ring containing from 5 to 14 ring atoms. The ring atoms of a carbocyclic ring are all carbon atoms. Aryl ring structures include one or more ring structures such as mono-, bi-, or tricyclic as well as fused aromatic carbocyclic moieties. Representative aryl rings include phenyl, anthracenyl, fluorenyl, indenyl, azulenyl, phenanthrenyl, naphthyl and the like. An aryl ring can be unsubstituted or substituted.

As used herein, unless otherwise specified the term “heteroaryl” means a carbocyclic aromatic ring containing from 5 to 14 ring atoms, wherein at least one of the carbocyclic ring atoms is replaced with at least one heteroatom, preferably 1 to 3 heteroatoms, independently selected from nitrogen, oxygen, or sulfur. Heteroaryl ring structures include one or more ring structures such as mono-, bi-, or tricyclic as well as fused aromatic carbocyclic (i.e. benzo-fused) and heterocarbocyclic moities. Representative heteroaryl rings include triazolyl, tetrazolyl, oxadiazolyl, pyridyl, furanyl, benzofuranyl, thienyl (also referred to as thiophenyl), benzothienyl (also referred to as benzothiophenyl), benzoisoxazolyl, benzoisothiazolyl, quinolinyl, isoquinolinyl, pyrrolyl, indolyl, indazolyl, oxazolyl, benzoxazolyl, imidazolyl, benzimidazolyl, thiazolyl, benzothiazolyl, thiadiazolyl, isoxazolyl, pyrazolyl, isothiazolyl, pyridazinyl, pyrimidinyl, pyrazinyl, triazinyl, cinnolinyl, phthalazinyl, quinazolinyl, benzoquinazolinyl, acridinyl, and the like. A heteroaryl ring can be unsubstituted or substituted.

As used herein, unless otherwise specified the term “aryloxy” means a radical of the formula: —O-aryl, wherein aryl is as defined above. An aryloxy radical can be unsubstituted or substituted.

As used herein, unless otherwise specified the term “arylalkyl” means a radical of the formula: -(alkyl)-(aryl), wherein alkyl and aryl are defined above, including —(CH₂)phenyl, —(CH₂)₂-phenyl, —(CH₂)₃-phenyl, —CH(phenyl)₂, —C(phenyl)₃, —(CH₂)tolyl, —(CH₂)anthracenyl, —(CH₂)fluorenyl, —(CH₂)indenyl, —(CH₂)azulenyl, —(CH₂)naphthyl, and the like.

As used herein, unless otherwise specified the term “heteroarylalkyl” means a radical of the formula: -(alkyl)-(heteroaryl), wherein alkyl and heteroaryl are defined above, including —(CH₂)pyridyl, —(CH₂)₂pyridyl, —(CH₂)₃pyridyl, —CH(pyridyl)₂, —C(pyridyl)₃, (CH₂)triazolyl, —(CH₂)tetrazolyl, —(CH₂)oxadiazolyl, —(CH₂)furyl, —(CH₂)benzofuranyl, —(CH₂)thiophenyl, —(CH₂)benzothiophenyl, and the like.

As used herein, unless otherwise specified the term “arylalkyloxy” means a radical of the formula: —O-(alkyl)-(aryl), wherein alkyl and aryl are defined above, including —O—(CH₂)₂-phenyl, —O—(CH₂)₃-phenyl, —O—CH(phenyl)₂, —O—CH(phenyl)₃, —O—(CH₂)tolyl, —O—(CH₂)anthracenyl, —O—(CH₂)fluorenyl, —O—(CH₂)indenyl, —O—(CH₂)azulenyl, —O—(CH₂)naphthyl, and the like.

As used herein, unless otherwise specified the term “cycloalkyl” means a monocyclic or polycyclic saturated or partially unsaturated carbocyclic ring containing from 3 to 14 ring atoms. Cycloalkyl ring structures include one or more ring structures such as mono-, bi-, or tricyclic as well as fused saturated or aromatic carbocyclic moieties such as 5,6,7,8-tetrahydronaphthyl and the like. Examples of cycloalkyl rings include, but are not limited to, (C₃-C₇)cycloalkyl groups, including cyclopropyl, cyclobutyl, cyclopentyl, cyclohexyl, and cycloheptyl, and saturated or partially unsaturated cyclic and bicyclic terpenes. A cycloalkyl group can be unsubstituted or substituted. Preferably, the cycloalkyl group is a monocyclic ring or bicyclic ring.

As used herein, unless otherwise specified the term “heterocyclyl” means a monocyclic or polycyclic saturated or partially unsaturated ring comprising carbon and hydrogen atoms, optionally having 1 to 4 multiple bonds, and the ring atoms contain at least one heteroatom, preferably 1 to 3 heteroatoms, independently selected from nitrogen, oxygen, and sulfur. Heterocyclyl ring structures include compounds having one or more ring structures such as mono-, bi-, or tricylic compounds. Preferably, the heterocyclyl group is a monocyclic ring or bicyclic ring. Representative heterocycles include, but are not limited to morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like. A heterocyclyl ring can be unsubstituted or substituted.

As used herein, unless otherwise specified the term “cycloalkyloxy” means a radical of the formula: —O-cycloalkyl, wherein cycloalkyl is defined above, including, but not limited to —O-cyclopropyl, —O-cyclobutyl, —O-cyclopentyl, —O-cyclohexyl, —O-cycloheptyl and the like.

As used herein, unless otherwise specified the term “cycloalkylalkoxy” means a radical of the formula: —O-(alkyl)-(cycloalkyl), wherein cycloalkyl and alkyl are defined above.

As used herein, unless otherwise specified the term “aminoalkoxy” means a radical of the formula: —O-(alkyl)-NH₂, wherein alkyl is defined above, including, but not limited to —O—CH₂—NH₂, —O—(CH₂)₂—NH₂, —O—(CH₂)₃—NH₂, —O—(CH₂)₄—NH₂, —O—(CH₂)₅—NH₂, and the like.

As used herein, unless otherwise specified the term “alkylamino” means a radical of the formula: —NH(alkyl) or —N(alkyl)(alkyl), wherein alkyl is defined above, including, but not limited to —NHCH₃, —NHCH₂CH₃, —NH(CH₂)₂CH₃, —NH(CH₂)₃CH₃, —NH(CH₂)₄—CH₃, —NH(CH₂)₅CH₃, —N(CH₃)₂, —N(CH₂CH₃)₂, —N((CH₂)₂CH₃)₂, —N(CH₃)(CH₂CH₃), and the like.

As used herein, unless otherwise specified the term “arylamino” means a radical of the formula: —NH(aryl), wherein aryl is defined above, including, but not limited to —NH(phenyl), —NH(tolyl), —NH(anthracenyl), —NH(fluorenyl), —NH(indenyl), —NH(azulenyl), —NH(pyridinyl), —NH(naphthyl), and the like.

As used herein, unless otherwise specified the term a radical of the formula: “arylalkylamino” means —NH-(alkyl)-(aryl), wherein alkyl and aryl are defined above, including —NH—CH₂— (phenyl), —NH—CH₂— (tolyl), —NH—CH₂— (anthracenyl), —NH—CH₂-(fluorenyl), —NH—CH₂— (indenyl), —NH—CH₂— (azulenyl), —NH—CH₂— (pyridinyl), —NH—CH₂-(naphthyl), —NH—(CH₂)₂-(phenyl) and the like.

As used herein, unless otherwise specified the term “cycloalkylamino” means a radical of the formula: —NH-(cycloalkyl), wherein cycloalkyl is defined above, including —NH-cyclopropyl, —NH-cyclobutyl, —NH-cyclopentyl, —NH-cyclohexyl, —NH-cycloheptyl, and the like.

As used herein, unless otherwise specified the term “aminoalkyl” means a radical of the formula: -(alkyl)-NH₂, wherein alkyl is defined above, including —CH₂—NH₂, —(CH₂)₂—NH₂, —(CH₂)₃—NH₂, —(CH₂)₄—NH₂, —(CH₂)₅—NH₂ and the like.

As used herein, unless otherwise specified the term “alkylaminoalkyl” means a radical of the formula: -(alkyl)-NH(alkyl) or -(alkyl)-N(alkyl)(alkyl), wherein each “alkyl” is independently an alkyl group defined above, including —CH₂—NH—CH₃, —CH₂—NHCH₂CH₃, —CH₂—NH(CH₂)₂CH₃, —CH₂—NH(CH₂)₃CH₃, —CH₂—NH(CH₂)₄—CH₃, —CH₂—NH(CH₂)₅CH₃, —(CH₂)₂—NH—CH₃, —CH₂—N(CH₃)₂, —CH₂—N(CH₂CH₃)₂, —CH₂—N((CH₂)₂CH₃)₂, —CH₂—N(CH₃)(CH₂CH₃), —(CH₂)₂—N(CH₃)₂, and the like.

Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. DETAILED DESCRIPTION

Presented herein are methods for treating MMA (e.g., mut⁰ MMA, mut⁻ MMA, cblA MMA, or cblB MMA) in which at least one allele of a gene associated with MMA (e.g., the MUT, MMAA, or MMAB gene) contains a mutation (e.g., nonsense mutation) that results in a premature stop codon in RNA encoded by an allele of the gene associated with MMA. Unless specified otherwise, as used hereinafter, MMA includes at least one allele of a gene associated with mut⁰ MMA, mut⁻ MMA, cblA MMA, and cblB MMA in which at least one allele of the MUT, MMAA, or MMAB gene contains a mutation (e.g., nonsense mutation) that results in a premature stop codon in RNA encoded by an allele of the MUT, MMAA or MMAB gene. In one aspect, the methods for treating MMA involve the administration of a Compound, as a single-agent therapy, to a patient in need thereof. In a specific embodiment, presented herein is a method for treating MMA, comprising administering to a patient in need thereof an effective amount of a Compound, as a single agent. In another embodiment, presented herein is a method for treating MMA, comprising administering to a patient in need thereof a pharmaceutical composition comprising a Compound, as the single active ingredient, and a pharmaceutically acceptable carrier, excipient or vehicle.

In certain embodiments, provided herein are Compounds for use in the methods recited herein.

In certain embodiments, provided herein are Compounds for use in the treatment of a disease or disorder recited herein, such as MMA.

In other embodiments, provided herein are Compounds for use in the treatment of a disease or disorder recited herein, such as MMA associated with a mutation in at least one allele of the MUT, MMAA (cblA) or MMAB (cblB) gene that results in a premature stop codon in RNA encoded by an allele of the MUT, MMAA or MMAB gene.

In other embodiments, provided herein are Compounds for use in the treatment of a disease or disorder recited herein, such as MMA associated with a mutation in at least one allele of the MUT gene that results in a premature stop codon in RNA encoded by an allele of the MUT gene.

In other embodiments, provided herein are Compounds for use in the treatment of a disease or disorder recited herein, such as MMA associated with a mutation in at least one allele of the MMAA gene that results in a premature stop codon in RNA encoded by an allele of the MMAA gene.

In other embodiments, provided herein are Compounds for use in the treatment of a disease or disorder recited herein, such as MMA associated with a mutation in at least one allele of the MMAB gene that results in a premature stop codon in RNA encoded by an allele of the MMAB gene.

In another aspect, the methods for treating MMA involve the administration of a Compound to a patient with at least one allele of a gene(s) associated with MMA containing one or more of the nucleotide changes identified in Table 1, Table 2, or Table 3 that result in a premature stop codon in RNA encoded by an allele of the gene associated with MMA. In a specific embodiment, provided herein is a method for treating MMA, comprising administering a Compound to a patient with at least one allele of the MUT gene that contains one or more of the nucleotide changes identified in Table 1 that result in a premature stop codon in RNA encoded by an allele of the MUT gene. In another specific embodiment, provided herein is a method for treating MMA, comprising administering a Compound to a patient with at least one allele of the MMAA gene that contains one or more of the nucleotide changes identified in Table 2 that result in a premature stop codon in RNA encoded by an allele of the MMAA gene. In another specific embodiment, provided herein is a method for treating MMA, comprising administering a Compound to a patient with at least one allele of the MMAB gene that contains one or more of the nucleotide changes identified in Table 3 that result in a premature stop codon in RNA encoded by an allele of the MMAB gene.

In another aspect, the methods for treating MMA involve the administration of a Compound in combination with another therapy (e.g., one or more additional therapies that do not comprise a Compound, or that comprise a different Compound) to a patient in need thereof. Such methods may involve administering a Compound prior to, concurrent with, or subsequent to administration of the additional therapy. In certain embodiments, such methods have an additive or synergistic effect. In a specific embodiment, presented herein is a method for treating MMA, comprising administering to a patient in need thereof an effective amount of a Compound and an effective amount of another therapy. Examples of such other therapies include, but are not limited to, cobalamin supplements, camitine supplements and antibiotics. In another specific embodiment, presented herein is a method for treating MMA, comprising administering to a patient in need thereof an effective amount of a Compound and maintaining a low-protein diet.

In certain embodiments, the concentration of methylmalonic acid (MMacid) in biological specimens (e.g., blood, plasma, serum, cerebral spinal fluid, urine, or any other biofluids) of a patient is monitored before, during and/or after a course of treatment involving the administration of a Compound or a pharmaceutical composition thereof to the patient. In certain embodiments, the concentration of methylcitrate in biological specimens (e.g., urine, blood, plasma, serum, cerebral spinal fluid, or any other biofluids) of a patient is monitored before, during and/or after a course of treatment involving the administration of a Compound or a pharmaceutical composition thereof to the patient. In certain embodiments, the concentration of propionylcarnitine in biological specimens (e.g., blood, plasma, serum, cerebral spinal fluid, urine, or any other biofluids) of a patient is monitored before, during and/or after a course of treatment involving the administration of a Compound or a pharmaceutical composition thereof to the patient. In certain embodiments, erythrocyte odd long-chain fatty acids (OLCFAs) levels are monitored before, during and/or after a course of treatment involving the administration of a Compound or a pharmaceutical composition thereof to a patient. In certain embodiments, the urinary urea:MMacid ratio is monitored before, during and/or after a course of treatment involving the administration of a Compound or a pharmaceutical composition thereof to a patient. The dosage, frequency and/or length of administration of a Compound or a pharmaceutical composition thereof to a patient may be modified as a result of the concentration of MMacid, methylcitrate, or propionylcarnitine, erythrocyte odd long-chain fatty acids (OLCFAs) levels, or the urinary urea:MMacid ratio. Alternatively, changes in one or more of these monitoring parameters (e.g., concentration of MMacid, methylcitrate, or propionylcarnitine, erythrocyte odd long-chain fatty acids (OLCFAs) levels, or the urinary urea.MMacid ratio) might indicate that the course of treatment involving the administration of the Compound or pharmaceutical composition thereof is effective in treating MMA.

In a specific embodiment, presented herein is a method for treating MMA, comprising: (a) administering to a patient in need thereof one or more doses of a Compound or a pharmaceutical composition thereof; and (b) monitoring the concentration of MMacid, methylcitrate, or propionylcarnitine (e.g., detected in biological specimens such as plasma, serum, cerebral spinal fluid, urine, or other biofluids), erythrocyte odd long-chain fatty acids (OLCFAs) levels, or the urinary urea:MMacid ratio before and/or after step (a). In certain embodiments, step (b) comprises monitoring the concentration of MMacid. In other embodiments, step (b) comprises monitoring the concentration of MMacid, methylcitrate and/or propionylcarnitine. In certain embodiments, the monitoring step (b) is carried out before and/or after a certain number of doses (e.g., 1, 2, A, 6, 8, 10, 12, 14, 15, or 20 doses, or more doses; or 2 to 4, 2 to 8, 2 to 20 or 2 to 30 doses) or after a certain time period (e.g., 1, 2, 3, 4, 5, 6, or 7 days; or 1, 2, 3, 4, 5, 10, 15, 20, 30, 40, 45, 48, or 50 weeks) of administering the Compound. In certain embodiments, one or more of these monitoring parameters are detected prior to administration of the Compound or pharmaceutical composition thereof. In specific embodiments, a decrease in the concentration of MMacid, methylcitrate, or propionylcamitine, or a decrease in erythrocyte odd long-chain fatty acids (OLCFAs) levels following administration of the Compound or pharmaceutical composition thereof indicates that the course of treatment is effective for treating MMA. In specific embodiments, an increase in the urinary urea:MMacid ratio following administration of the Compound or pharmaceutical composition thereof indicates that the course of treatment is effective for treating MMA. In some embodiments, a change in the concentration of MMacid, methylcitrate, or propionylcamitine, a change in the levels of erythrocyte odd long-chain fatty acids (OLCFAs) levels, or a change in the urinary urea:MMacid ratio following administration of the Compound or pharmaceutical composition thereof may indicate that the dosage, frequency and/or length of administration of the Compound or a pharmaceutical composition thereof may should be adjusted (e.g., increased, reduced or maintained).

The concentration of MMacid, methylcitrate, or propionylcamitine, erythrocyte odd long-chain fatty acids (OLCFAs) levels, or the urinary urea:MMacid ratio of a patient may be detected by any technique known to one of skill in the art. In certain embodiments, the method for detecting the concentration of MMacid, methylcitrate, or propionylcarnitine in a patient involves obtaining a tissue or fluid sample from the patient and detecting the concentration of MMacid, methylcitrate, propionylcarnitine or urea in the biological sample (e.g., from plasma serum sample, cerebral spinal fluid, urine, or other biofluids) that has been subjected to certain types of treatment (e.g., centrifugation) and detection by use of, e.g., standard gas chromatography/mass spectroscopy (GC/MS) stable-isotope dilution methods, positive chemical ionization gas chromatography mass spectrometry (CI GC-MS) spectroscopic techniques (e.g., UV spectroscopy) or high pressure liquid chromatography (HPLC). Erythrocyte odd long-chain fatty acids levels can, e.g., be measured by extracting fatty acids in erythrocyte membranes with a mixture of chloroform and methanol (2:1, by volume), collecting them in heptane, and injecting them onto a Varian CP7420 100-m capillary column with a Hewlett-Packard 5890 gas chromatograph equipped with an HP6890A autosampler. The initial temperature of 190° C. can be increased to 240° C. over 50 min to separate fatty acids.

In specific embodiments, the methods for treating MMA provided herein alleviate or manage one, two or more symptoms associated with MMA. Alleviating or managing one, two or more symptoms of MMA may be used as a clinical endpoint for efficacy of a Compound for treating MMA. In some embodiments, the methods for treating MMA provided herein reduce the duration and/or severity of one or more symptoms associated with MMA. In some embodiments, the methods for treating MMA provided herein inhibit the onset, progression and/or recurrence of one or more symptoms associated with MMA. In some embodiments, the methods for treating MMA provided herein reduce the number of symptoms associated with MMA. In certain embodiments, the methods for treating MMA provided herein inhibit or reduce the progression of one or more symptoms associated therewith.

Symptoms associated with MMA include, but are not limited to: apnea, hyperammonemia, metabolic acidosis, lethargy, vomiting, dehydration, hypotonia, hypoglycemia, repeated yeast infections, renal impairment, mental retardation, developmental delays, seizures, movement disorders, progressive encephalopathy, facial dysmorphism (e.g., high forehead, broad nasal bridge, epicanthal folds, long smooth philtrum, or triangular mouth), stroke, skin lesions (e.g., moniliasis), occasional hepatomegaly, acute onset of choreoathetosis, dystonia, dysphagia, dysarthria, growth problems (e.g., growth failure), kidney disease or failure, tissue damage, feeding problems, cognitive disabilities, metabolic attacks triggered by common infections and reduced glomerular filtration rate (GFR).

In specific embodiments, the methods for treating MMA provided herein reduce or eliminate one, two, or more of the following: metabolic acidosis, developmental delays, movement disorders, metabolic decompensation episodes (e.g., frequency and/or numbers of episodes), skin lesions, hypotonia, seizures, and renal impairment, associated with MMA. In some embodiments, the methods for treating MMA provided herein improve renal function, development, cognitive ability and movement in a patient diagnosed with MMA.

In specific embodiments, the methods for treating MMA provided herein reduce hospitalization (e.g., the frequency or duration of hospitalization) of a patient diagnosed with MMA. In some embodiments, the methods for treating MMA provided herein reduce hospitalization length of a patient diagnosed with MMA. In certain embodiments, the methods for treating MMA provided herein decrease the hospitalization rate.

In specific embodiments, provided herein are methods for treating MMA, comprising administering to a human having a mutation (e.g., nonsense mutation) in at least one allele of the MMAA (cblA) or MMAB (cblB) gene that results in a premature stop codon in RNA encoded by an allele of the MMAA or MMAB gene an effective amount of a Compound (e.g., a compound of formula I, formula II, formula III or 3-[5-(2-Fluoro-phenyl)-[1,2,4]oxadiazol-3-yl]-benzoic acid or a pharmaceutically acceptable salt, hydrate, solvate or stereoisomer thereof).

In specific embodiments, provided herein are methods for treating MMA, comprising administering to a human having a mutation (e.g., nonsense mutation) in at least one allele of the MUT gene that results in a premature stop codon in RNA encoded by an allele of the MUT gene an effective amount of a Compound (e.g., a compound of formula I, formula II, formula III or 3-[5-(2-Fluoro-phenyl)-[1,2,4]oxadiazol-3-yl]-benzoic acid or a pharmaceutically acceptable salt, hydrate, solvate or stereoisomer thereof).

In certain embodiments, the methods provided herein increase the survival of a patient diagnosed with MMA. In particular embodiments, the methods for treating MMA provided herein reduce the mortality of subjects diagnosed with MMA. In particular embodiments, the methods for treating MMA provided herein increase symptom-free survival of MMA patients. In some embodiments, the methods for treating MMA provided herein do not cure MMA in patients, but prevent the progression or worsening of the disease. In specific embodiments, the methods for treating MMA provided herein enhance or improve the therapeutic effect of another therapy.

In specific embodiments, the methods for treating MMA achieve one or more of the clinical endpoints set forth in the working examples in Section 6 et seq. In particular embodiments, the methods for treating MMA achieve one or more of the following: (i) a decrease in the concentration of MMacid in biological specimens (e.g., plasma, serum, cerebral spinal fluid, urine, or any other biofluids); (ii) a decrease in the concentration of propionylcarnitine in biological specimens (e.g., plasma, serum, cerebral spinal fluid, urine, or any other biofluids); (iii) a decrease in the concentration of methylcitrate in biological specimens (e.g., plasma, serum, cerebral spinal fluid, urine, or any other biofluids); (iv) a decrease in erythrocyte OLCFAs levels; (vi) an increase in the urinary urea:MMacid ratio; (vii) an increase in cellular enzyme activity in, e.g., fibroblasts or lymphocytes; (vii) an improvement in developmental or cognitive ability; (viii) a decrease in hospitalization; (ix) a decrease in laboratory abnormalities; (x) a decrease in metabolic decompensation episodes, characterized by, e.g., vomiting, hyptonia, and alteration of consciousness associated with metabolic acidosis and hyperammoneia; (xi) an improvement in renal function; (xii) an improvement in movement; and (xiii) improvement in quality of life as assessed by methods well known in the art, e.g., questionnaires.

In particular embodiments, the methods for treating MMA provided herein reduce the concentration of plasma MMacid in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a Compound, as assessed by methods well known in the art or described herein. In certain embodiments, the methods for treating MMA provided herein reduce the concentration of urinary MMacid in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a Compound, as assessed by methods well known in the art or described herein.

In specific embodiments, the methods for treating MMA provided herein reduce the concentration of a metabolite of MMacid in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a Compound, as assessed by methods well known in the art or described herein. In some embodiments, the methods for treating MMA provided herein reduce the concentration of plasma propionylcamitine in a subject by at least about 5%, 10%, 15%, 20%, 25%, 30%, 35%, 40%, 45%, 50%, 55%, 60%, 65%, 80%, 85%, 90%, 95%, or 100%, or in a range of from 5% to 50%, 10% to 50%, 20% to 50%, 20% to 75%, 25% to 75%, 25% to 90% or 10% to 99% relative to the respective concentration prior to administration of a Compound, as assessed by methods well known in the art or described herein. In certain embodiments, the methods for treating MMA provided herein reduce the concentration of urinary methylcitrate in a subject by at least about 5%, 10%, 15%, 20%, 25%, 30%, 35%, 40%, 45%, 50%, 55%, 60%, 65%, 80%, 85%, 90%, 95%, or 100%, or in a range of from 10% to 50%, 20% to 50%, 20% to 75%, 25% to 75%, 25% to 90% or 10% to 99% relative to the respective concentration prior to administration of a Compound, as assessed by methods well known in the art or described herein.

In certain embodiments, the methods for treating MMA provided herein reduce the erythrocyte OLCFA levels in a subject by at least about 5%, 10%, 15%, 20%, 25%, 30%, 35%, 40%, 45%, 50%, 55%, 60%, 65%, 80%, 85%, 90%, 95%, or 100%, or in a range of from 10% to 50%, 20% to 50%, 20% to 75%, 25% to 75%, 25% to 90% or 10% to 99% relative to the respective concentration prior to administration of a Compound, as assessed by methods well known in the art or described herein. In some embodiments, the methods for treating MMA provided herein increase the urinary urea:MMacid ratio in a subject by at least about 5%, 10%, 15%, 20%, 25%, 30%, 35%, 40%, 45%, 50%, 55%, 60%, 65%, 80%, 85%, 90%, 95%, or 100%, or in a range of from 10% to 50%, 20% to 50%, 20% to 75%, 25% to 75%, 25% to 90% or 10% to 99% relative to the respective concentration prior to administration of a Compound, as assessed by methods well known in the art or described herein.

In specific embodiments, the methods for treating MMA provided herein increase the cellular enzyme activity in a subject by at least about 5%, 10%, 15%, 20%, 25%, 30%, 35%, 40%, 45%, 50%, 55%, 60%, 65%, 80%, 85%, 90%, 95%, or 100%, or in a range of from 10% to 50%, 20% to 50%, 20% to 75%, 25% to 75%, 25% to 90% or 10% to 99% relative to the respective concentration prior to administration of a Compound, as assessed by methods well known in the art or described herein. In certain embodiment, the increase in cellular enzyme activity is determined by obtaining cells (e.g., fibroblasts or lymphocytes) from the subject, culturing the cells in the presence or absence of a Compound, and comparing the cellular enzyme activity in the presence of the Compound to the cellular enzyme activity in the absence of the Compound. Techniques for measuring cellular enzyme activity are known in the art and described herein (see, e.g., Section 6, infra).

In some aspects, the methods for treating MMA provided herein improve or developmental or cognitive function in a subject. Such improvements in developmental or cognitive function may be as assessed by, e.g., the Bayley Scale of Infant Development, Wechsler Preschool and Primary Scale of Intelligence (WIPPSI), Wechsler Intelligence Scale for Children (WISC) or Wechsler Adult Intelligence Scale (WAIS). In a specific embodiment, an improvement in developmental or cognitive function may be assessed using the methods provided in the working examples in Section 6 et seq.

In some aspects, the methods for treating MMA provided herein improve control of muscle contractions by a subject as assessed by methods well known in the art, e.g., the Burke-Fahn-Marsden rating scale. In certain aspects, the methods for treating MMA provided herein decrease the occurrence of metabolic decompensation episodes, characterized by, e.g., vomiting, hypotonia, and alteration in consciousness.

In some aspects, the methods for treating MMA provided herein improve renal function. In certain embodiments, the methods for treating MMA provided herein decrease the need for kidney transplant, liver transplant or both.

In some aspects, the methods for treating MMA provided herein decrease the requirement for hospitalization. In certain embodiments, the methods for treating MMA provided herein decrease the length and/or frequency of hospitalization.

In certain aspects, provided herein are methods for the production of a functional readthrough protein from a MUT gene comprising a mutation (e.g., nonsense mutation) that results in a premature stop codon in RNA encoded by the MUT gene, the methods comprising contacting a cell containing such a MUT gene with a Compound. In a specific embodiment, provided herein is a method for the production of a functional readthrough protein from a MUT gene comprising a mutation (e.g., nonsense mutation) at one or more of the positions of the gene identified in Table 1 below that results in a premature stop codon in RNA encoded by the MUT gene, the method comprising contacting a cell containing such a MUT gene with a Compound in an amount effective to produce the functional readthrough protein. In some embodiments, the functional readthrough protein produced has an amino acid residue other than the amino acid residue found at the same location in the corresponding wild-type protein.

In certain aspects, provided herein are methods for the production of a functional readthrough protein, the methods comprising administering a Compound to a patient with at least one allele of the MUT gene comprising a mutation (e.g., nonsense mutation) that results in a premature stop codon in RNA encoded by an allele of the MUT gene. In a specific embodiment, provided herein is a method for the production of a functional readthrough protein, the method comprising administering a Compound to a patient with at least one allele of the MUT gene comprising a mutation (e.g., nonsense mutation) at one or more of the positions identified in Table 1 that results in a premature stop codon in RNA encoded by an allele of the MUT gene. In another specific embodiment, provided herein is a method for the production of a functional readthrough protein, the method comprising administering an effective amount of a Compound to a patient with at least one allele of the MUT gene comprising a mutation (e.g., nonsense mutation) at one or more of the positions identified in Table 1 that results in a premature stop codon in RNA encoded by an allele of the MUT gene, wherein the effective amount of the Compound is an amount that results in the production of the functional readthrough protein. The amount of the functional readthrough protein or the production of the functional readthrough protein may be assessed using techniques known to those skilled in the art, including, but not limited to, cell culture techniques or animal models. In certain embodiments, the amount of functional readthrough protein produced is sufficient to ameliorate or reduce one or more of the symptoms associated with MMA.

TABLE 1¹

| Location | Nucleotide change² | Amino Acid Substitution |
|---|---|---|
| Exon 5 | c.1025 C > A | p.S342X |
| Exon 2 | c.19 C > T | p.Q7X |
| Exon 2 | c.52C > T | p.Q18X |
| Exon 6 | c.1237 C > T | p.Q413X |
| Exon 3 | c.682 C > T | p.R228X |
| Exon 2 | c.160 A > T | p.K54X |
| Exon 6 | c.1207 C > T | p.R403X |
| Exon 6 | c.1240 G > T | p.E414X |
| Exon 7 | c.1423 C > T | p.R474X |
| Exon 7 | c.1399 C > T | p.R467X |
| Exon 8 | c.1531 C > T | p.R511X |
| Exon 8 | c.454 C > T | p.R152X |
| Exon 8 | c.397 C > T | p.Q133X |
| Exon 8 | c.433 C > T | p.R145X |
| Exon 8 | c.358 C > T | p.Q120X |

¹Table 1 was adapted from Acquaviva et al., 2005, Human Mutation 25: 167-176 and Martinez et al., 2005, Molecular Genetics and Metabolism 84: 317-325.
²The DNA mutation numbering is based on cDNA reference sequence (GenBank Accession No. M65131.1) considering nucleotide +1 as the A of the ATG.

In certain aspects, provide herein are methods for the production of a functional readthrough protein from a MMAA gene comprising a mutation (e.g., nonsense mutation) that results in a premature stop codon in RNA encoded by the MMAA gene, the methods comprising contacting a cell containing such a MMAA gene with a Compound. In a specific embodiment, provided herein is a method for the production of a functional readthrough protein from a MMAA gene comprising a mutation (e.g., nonsense mutation) at one or more of the positions of the gene identified in Table 2 below that results in a premature stop codon in RNA encoded by the gene, the method comprising contacting a cell containing such a MMAA gene with a Compound. In some embodiments, the functional readthrough protein produced has an amino acid residue other than the amino acid residue found at the same location in the corresponding wild-type protein.

In certain aspects, provided herein are methods for the production of a functional readthrough protein, the methods comprising administering a Compound to a patient with at least one allele of the MMAA gene comprising a mutation (e.g., nonsense mutation) that results in a premature stop codon in RNA encoded by an allele of the MMAA gene. In a specific embodiment, provided herein is a method for the production of a functional readthrough protein, the method comprising administering a Compound to a patient with at least one allele of the MMAA gene comprising a mutation (e.g., nonsense mutation) at one or more of the positions identified in Table 2 that results in a premature stop codon in RNA encoded by an allele of the MMAA gene. In another specific embodiment, provided herein is a method for the production of a functional readthrough protein, the method comprising administering an effective amount of a Compound to a patient with at least one allele of the MMAA gene comprising a mutation (e.g., nonsense mutation) at one or more of the positions identified in Table 2 that results in a premature stop codon in RNA encoded by an allele of the MMAA gene, wherein the effective amount of the Compound is an amount that results in the production of the functional readthrough protein. The amount of the functional readthrough protein or the production of the functional readthrough protein may be assessed using techniques known to those skilled in the art, including, but not limited to, cell culture techniques or animal models. In certain embodiments, the amount of functional readthrough protein produced is sufficient to ameliorate or reduce one or more of the symptoms associated with MMA.

TABLE 2¹

| Location | Nucleotide Change | Amino Acid Substitution |
|---|---|---|
| Exon 4 | c.812_813 dupAG | p.L272fs V288X |
| Exon 3 | c.594 dupT | p.E199fsE199X |
| Exon 2 | c.450dupG | p.P151fsE169X |
| Exon 2 | c.385 C > T | p.Q120X |
| Exon 2 | c.433 C > T | p.R145X |

¹Table 2 was adapted from Martinez et al., 2005, Molecular Genetics and Metabolism 84: 317-325.
²The DNA mutation numbering is based on cDNA reference sequence (e.g., GenBank Accession No. NM_172250) considering nucleotide +1 as the A of the ATG.

In certain aspects, provided herein are methods for the production of a functional readthrough protein from a MMAB gene comprising a mutation (e.g., nonsense mutation) that results in a premature stop codon in RNA encoded by the MMAB gene, the methods comprising contacting a cell containing such a MMAB gene with a Compound. In a specific embodiment, provided herein is a method for the production of a functional readthrough protein from a MMAB gene comprising a mutation (e.g., nonsense mutation) at one or more of the positions of the gene identified in Table 3 below that results in a premature stop codon in RNA encoded by the gene, the method comprising contacting a cell containing such a MMAB gene with a Compound. In some embodiments, the functional readthrough protein produced has an amino acid residue other than the amino acid residue found at the same location in the corresponding wild-type protein.

In certain aspects, provided herein are methods for the production of a functional readthrough protein, the methods comprising administering a Compound to a patient with at least one allele of the MMAB gene comprising a mutation (e.g., nonsense mutation) that results in a premature stop codon in RNA encoded by an allele of the MMAB gene. In a specific embodiment, provided herein is a method for the production of a functional readthrough protein, the method comprising administering a Compound to a patient with at least one allele of the MMAB gene comprising a mutation (e.g., nonsense mutation) at one or more of the positions identified in Table 3 that results in a premature stop codon in RNA encoded by an allele of the MMAB gene. In another specific embodiment, provided herein is a method for the production of a functional readthrough protein, the method comprising administering an effective amount of a Compound to a patient with at least one allele of the MMAB gene comprising a mutation (e.g., nonsense mutation) at one or more of the positions identified in Table 3 that results in a premature stop codon in RNA encoded by an allele of the MMAB gene, wherein the effective amount of the Compound is an amount that results in the production of the functional readthrough protein. The amount of the functional readthrough protein or the production of the functional readthrough protein may be assessed using techniques known to those skilled in the art, including, but not limited to, cell culture techniques or animal models. In certain embodiments, the amount of functional readthrough protein produced is sufficient to ameliorate or reduce one or more of the symptoms associated with MMA.

TABLE 3¹

| Location | Nucleotide Change | Amino Acid Substitution |
|---|---|---|
| Intron 3 | c.291-1G > A | p.G97fsD218X |

¹Table 3 was adapted from Martinez et al., 2005, Molecular Genetics and Metabolism 84: 317-325.
²The DNA mutation numbering is based on cDNA reference sequence (e.g., GenBank Accession No. NM_052845) considering nucleotide +1 as the A of the ATG.

In certain aspects, provided herein are methods for treating MMA, the methods comprising administering a Compound to a patient having a gene associated with MMA that when transcribed produces a mRNA containing a premature stop codon. In a specific embodiment, provided herein is a method for treating MMA, the method comprising administering a Compound to a patient having a MUT gene that when transcribed produces a mRNA containing a premature stop codon. In another specific embodiment, provided herein is a method for treating MMA, the method comprising administering a Compound to a patient having a MMAA gene that when transcribed produces a mRNA containing a premature stop codon. In another specific embodiment, provided herein is a method for treating MMA, the method comprising administering a Compound to a patient having a MMAB gene that when transcribed produces a mRNA containing a premature stop codon.

In some aspects, cells (e.g., fibroblasts or lymphocytes) from an MMA patient are cultured in the presence or absence of a Compound to assess responsiveness of the subject to the treatment methods provided herein. In a specific embodiment, cells (e.g., fibroblasts or lymphocytes) from an MMA patient are cultured in the presence or absence of a Compound and the cellular enzyme activity (e.g., MCM activity or the activity of an enzyme required for the active form of AdoCbl) in the presence of the Compound relative to the cellular enzyme activity in the absence of the Compound is determined using techniques known to one skilled in the art or as described herein (see, e.g., Section 6 et. seq., infra). In accordance with such an embodiment, an increase in cellular enzyme activity in the presence of the Compound relative to the cellular enzyme activity in the absence of the Compound indicates that the cells from the MMA patient are responsive to the Compound, which in turn indicates that the MMA patient will be responsive to treatment with the Compound.

### 4.1 Compounds

Provided herein are 1,2,4-oxadiazole benzoic acid compounds of formula I:

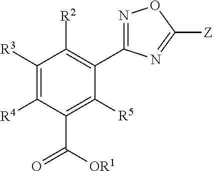

and pharmaceutically acceptable salts, hydrates, solvates and stereoisomers, including enantiomers, diastereomers, racemates or mixtures of stereoisomers, thereof wherein:

Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl;

R¹ is hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, —(CH₂CH₂O)ₙR⁶ or a biohydrolyzable group;

R², R³, R⁴, R⁵ and R⁶ are independently hydrogen, hydroxyl, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen, CF₃, OCF₃, OCHF₂, CN, COOH, COOR⁷, SO₂R⁷, NO₂, NH₂, or N(R⁷)₂;

each occurrence of R⁷ is independently hydrogen, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen or CF₃; and

n is an integer from 1 to 7.

In one embodiment, R⁶ is hydrogen or substituted or unsubstituted alkyl.

In one embodiment, provided herein are compounds of formula I wherein R¹ is H.

In another embodiment, provided herein are compounds of formula I wherein R¹ is a biohydrolyzable group other than H.

In one embodiment, provided herein are 1,2,4-oxadiazole benzoic acid compounds of the formula II:

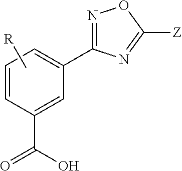

and pharmaceutically acceptable salts, hydrates, solvates and stereoisomers, including enantiomers, diastereomers, racemates or mixtures of stereoisomers, thereof wherein:

Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl; and R is hydrogen or halogen.

In one embodiment R is the halogen, fluorine. In another embodiment, R is hydrogen.

In certain embodiments, provided herein are compounds of formula I or II wherein Z is p-Tolyl; (4-Chloromethyl-phenyl); (2-Chloro-pyridin-3-yl); (2-Fluoro-phenyl); (3,4-Difluoro-phenyl); (4-Methoxy-phenyl); Benzo[1,3]dioxol-yl; (4-Ethyl-phenyl); o-Tolyl; (2-Chloro-phenyl); (3-Methyl-thiophen-2-yl); Benzo[b]thiophen-2-yl; (3-Fluoro-phenyl); (4-tert-Butyl-phenyl); (2-Methoxy-phenyl); (2,5-Difluoro-phenyl); Thiophen-2-yl; (2,4-Difluoro-phenyl); (3-Chloro-phenyl); m-Tolyl; (4-Trifluoromethyl-phenyl); (4-Fluoro-phenyl); (3-Methoxy-phenyl); Phenyl; (2,6-Difluoro-phenyl); (2,5-Dimethyl-furan-3-yl); (4-Pyrrol-1-yl-phenyl); (3-Dimethylamino-phenyl); Biphenyl-4-yl; (4-Dimethylamino-phenyl); Benzo[2,1,3]oxadiazol-5-yl; (2-Trifluoromethyl-phenyl); (6-Chloro-pyridin-3-yl); (3,5-Bis-trifluoromethyl-phenyl); Furan-2-yl; (4-Nitro-phenyl); (3,4-Dimethoxy-phenyl); (3-Trifluoromethoxy-phenyl); Naphthalen-1-yl; Cyclohexyl; Pyridin-3-yl; Pyridin-4-yl; Cyclopentyl; Cyclopropyl; (4-Pentyloxy-phenyl); (3,4,5-Trimethoxy-phenyl); (4-Isobutyl-phenyl); Cyclobutyl; (1-Acetyl-piperidin-4-yl); Isoxazol-5-yl; [3-(2-Chloro-6-fluoro-phenyl)-5-methyl-isoxazol-4-yl]; [3-(2-Chloro-phenyl)-5-methyl-isoxazol-4-yl]; Isopropyl; tert-Butyl; Butyl; Propenyl; 4-Chloro-benzyl; 4-Chloro-phenoxymethyl; Benzyl; Methoxymethyl; 1-Phenyl-propyl; 4-Fluoro-benzyl; 3-Chloro-phenoxymethyl; 6-Chloro-pyridin-3-yl; Cyclopentylmethyl; 4-Methoxy-benzyl; 2,3-Difluoro-phenyl; 2-Fluoro-5-methyl-phenyl; 2-Methylsulfanyl-pyridin-3-yl; 2,2-Difluoro-benzo[1,3]dioxol-5-yl; 4-Chloro-2-fluoro-phenyl; 4-Bromo-2-fluoro-phenyl; 3-Fluoro-biphenyl-4-yl; 3-(2-Chloro-phenyl)-5-methyl-isoxazol-4-yl; 6-Pyrrolidin-1-yl-pyridin-3-yl; 6-Morpholin-4-yl-pyridin-3-yl; 3,4,5,6-Tetrahydro-2H-[1,2′]bipyridinyl-5′-yl; 2-Fluoro-6-hydroxy-phenyl; 4-Amino-phenyl; 4-Azido-phenyl; or 4-Benzyloxy-phenyl.

In one embodiment, provided herein are compounds of the formula III:

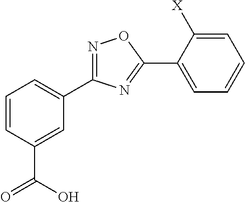

and pharmaceutically acceptable salts, hydrates, solvates and stereoisomers, including enantiomers, diastereomers, racemates or mixtures of stereoisomers, thereof wherein:

X is halogen, substituted alkyl or substituted or unsubstituted alkoxy.

Illustrative compounds provided herein include, but are not limited to,
3-(5-p-Tolyl-[1,2,4]oxadiazol-3-yl)-benzoic acid;
3-[5-(4-Chloromethyl-phenyl)-[1,2,4]oxadiazol-3-yl]-benzoic acid;
3-[5-(2-Chloro-pyridin-3-yl)-[1,2,4]oxadiazol-3-yl]-benzoic acid;
3-[5-(2-Fluoro-phenyl)-[1,2,4]oxadiazol-3-yl]-benzoic acid;
3-[5-(3,4-Difluoro-phenyl)-[1,2,4]oxadiazol-3-yl]-benzoic acid;
3-[5-(4-Methoxy-phenyl)-[1,2,4]oxadiazol-3-yl]-benzoic acid;
3-(5-Benzo[1,3]dioxol-5-yl-[1,2,4]oxadiazol-3-yl)-benzoic acid;
3-[5-(4-Ethyl-phenyl)-[1,2,4]oxadiazol-3-yl]-benzoic acid;
3-(5-o-Tolyl-[1,2,4]oxadiazol-3-yl)-benzoic acid;
3-[5-(2-Chloro-phenyl)-[1,2,4]oxadiazol-3-yl]-benzoic acid;
3-[5-(3-Methyl-thiophen-2-yl)-[1,2,4]oxadiazol-3-yl]-benzoic acid;
3-(5-Benzo[b]thiophen-2-yl-[1,2,4]oxadiazol-3-yl)-benzoic acid;
3-[5-(3-Fluoro-phenyl)-[1,2,4]oxadiazol-3-yl]-benzoic acid;
3-[5-(4-tert-Butyl-phenyl)-[1,2,4]oxadiazol-3-yl]-benzoic acid;
3-[5-(2-Methoxy-phenyl)-[1,2,4]oxadiazol-3-yl]-benzoic acid;
3-[5-(2,5-Difluoro-phenyl)-[1,2,4]oxadiazol-3-yl]-benzoic acid;
3-(5-Thiophen-2-yl-[1,2,4]oxadiazol-3-yl)-benzoic acid;
3-[5-(2,4-Difluoro-phenyl)-[1,2,4]oxadiazol-3-yl]-benzoic acid;
3-[5-(3-Chloro-phenyl)-[1,2,4]oxadiazol-3-yl]-benzoic acid;
3-(5-m-Tolyl-[1,2,4]oxadiazol-3-yl)-benzoic acid;
3-[5-(4-Trifluoromethyl-phenyl)-[1,2,4]oxadiazol-3-yl]-benzoic acid;
3-[5-(4-Fluoro-phenyl)-[1,2,4]oxadiazol-3-yl]-benzoic acid;
3-[5-(3-Methoxy-phenyl)-[1,2,4]oxadiazol-3-yl]-benzoic acid;
3-(5-Phenyl-[1,2,4]oxadiazol-3-yl)-benzoic acid;
3-[5-(2,6-Difluoro-phenyl)-[1,2,4]oxadiazol-3-yl]-benzoic acid;
3-[5-(2,5-Dimethyl-furan-3-yl)-[1,2,4]oxadiazol-3-yl]-benzoic acid;
3-[5-(4-Pyrrol-1-yl-phenyl)-[1,2,4]oxadiazol-3-yl]-benzoic acid;
3-[5-(3-Dimethylamino-phenyl)-[1,2,4]oxadiazol-3-yl]-benzoic acid;
3-(5-Biphenyl-4-yl-[1,2,4]oxadiazol-3-yl)-benzoic acid;
3-[5-(4-Dimethylamino-phenyl)-[1,2,4]oxadiazol-3-yl]-benzoic acid;
3-(5-Benzo[2,1,3]oxadiazol-5-yl-[1,2,4]oxadiazol-3-yl)-benzoic acid;
3-[5-(2-Trifluoromethyl-phenyl)-[1,2,4]oxadiazol-3-yl]-benzoic acid;
3-[5-(6-Chloro-pyridin-3-yl)-[1,2,4]oxadiazol-3-yl]-benzoic acid;
3-[5-(3,5-Bis-trifluoromethyl-phenyl)-[1,2,4]oxadiazol-3-yl]-benzoic acid;
3-(5-Furan-2-yl-[1,2,4]oxadiazol-3-yl)-benzoic acid;
3-[5-(4-Nitro-phenyl)-[1,2,4]oxadiazol-3-yl]-benzoic acid;
3-[5-(3,4-Dimethoxy-phenyl)-[1,2,4]oxadiazol-3-yl]-benzoic acid;
3-[5-(3-Trifluoromethoxy-phenyl)-[1,2,4]oxadiazol-3-yl]-benzoic acid;
3-(5-Naphthalen-1-yl-[1,2,4]oxadiazol-3-yl)-benzoic acid;
3-(5-Cyclohexyl-[1,2,4]oxadiazol-3-yl)-benzoic acid;
3-(5-Pyridin-3-yl-[1,2,4]oxadiazol-3-yl)-benzoic acid;
3-(5-Pyridin-4-yl-[1,2,4]oxadiazol-3-yl)-benzoic acid;
3-(5-Cyclopentyl-[1,2,4]oxadiazol-3-yl)-benzoic acid;
3-(5-Cyclopropyl-[1,2,4]oxadiazol-3-yl)-benzoic acid;
3-[5-(4-Pentyloxy-phenyl)-[1,2,4]oxadiazol-3-yl]-benzoic acid;
3-[5-(3,4,5-Trimethoxy-phenyl)-[1,2,4]oxadiazol-3-yl]-benzoic acid;
3-[5-(4-Isobutyl-phenyl)-[1,2,4]oxadiazol-3-yl]-benzoic acid;
3-(5-Cyclobutyl-[1,2,4]oxadiazol-3-yl)-benzoic acid;
3-[5-(1-Acetyl-piperidin-4-yl)-[1,2,4]oxadiazol-3-yl]-benzoic acid;
3-(5-Isoxazol-5-yl-[1,2,4]oxadiazol-3-yl)-benzoic acid;
3-{5-[3-(2-Chloro-6-fluoro-phenyl)-5-methyl-isoxazol-4-yl]-[1,2,4]oxadiazol-3-yl}-benzoic acid;
3-(5-Isopropyl-[1,2,4]oxadiazol-3-yl)-benzoic acid;
3-(5-tert-Butyl-[1,2,4]oxadiazol-3-yl)-benzoic acid;
3-(5-Butyl-[1,2,4]oxadiazol-3-yl)-benzoic acid;
3-(5-Propenyl-[1,2,4]oxadiazol-3-yl)-benzoic acid;
3-[5-(4-Chloro-benzyl)-[1,2,4]oxadiazol-3-yl]-benzoic acid;
3-[5-(4-Chloro-phenoxymethyl)-[1,2,4]oxadiazol-3-yl]-benzoic acid;
3-(5-Benzyl-[1,2,4]oxadiazol-3-yl)-benzoic acid;
3-(5-Methoxymethyl-[1,2,4]oxadiazol-3-yl)-benzoic acid;
3-[5-(1-Phenyl-propyl)-[1,2,4]oxadiazol-3-yl]-benzoic acid;
3-[5-(4-Fluoro-benzyl)-[1,2,4]oxadiazol-3-yl]-benzoic acid;
3-[5-(3-Chloro-phenoxymethyl)-[1,2,4]oxadiazol-3-yl]-benzoic acid;
3-[5-(6-Chloro-pyridin-3-yl)-[1,2,4]oxadiazol-3-yl]-benzoic acid;
3-(5-Cyclopentylmethyl-[1,2,4]oxadiazol-3-yl)-benzoic acid;
3-[5-(4-Methoxy-benzyl)-[1,2,4]oxadiazol-3-yl]-benzoic acid;
3-[5-(2,3-Difluoro-phenyl)-[1,2,4]oxadiazol-3-yl]-benzoic acid;
3-[5-(2-Fluoro-5-methyl-phenyl)-[1,2,4]oxadiazol-3-yl]-benzoic acid;
3-[5-(2-Methylsulfanyl-pyridin-3-yl)-[1,2,4]oxadiazol-3-yl]-benzoic acid;
3-[5-(2,2-Difluoro-benzo[1,3]dioxol-5-yl)-[1,2,4]oxadiazol-3-yl]-benzoic acid;
4-Fluoro-3-[5-(4-fluoro-phenyl)-[1,2,4]oxadiazol-3-yl]-benzoic acid;
2-Fluoro-5-[5-(4-fluoro-phenyl)-[1,2,4]oxadiazol-3-yl]-benzoic acid;
3-[5-(4-Chloro-2-fluoro-phenyl)-[1,2,4]oxadiazol-3-yl]-benzoic acid;
3-[5-(4-Bromo-2-fluoro-phenyl)-[1,2,4]oxadiazol-3-yl]-benzoic acid;
3-[5-(3-Fluoro-biphenyl-4-yl)-[1,2,4]oxadiazol-3-yl]-benzoic acid;
3-{5-[3-(2-Chloro-phenyl)-5-methyl-isoxazol-4-yl]-[1,2,4]oxadiazol-3-yl}-benzoic acid;
3-[5-(4-Cyano-phenyl)-[1,2,4]oxadiazol-3-yl]-benzoic acid;
3-[5-(2-Fluoro-phenyl)-[1,2,4]oxadiazol-3-yl]-benzoic acid sodium salt;
3-[5-(4-Fluoro-phenyl)-[1,2,4]oxadiazol-3-yl]-benzoic acid methyl ester;
5-[5-(4-Fluoro-phenyl)-[1,2,4]oxadiazol-3-yl]-2-methoxy-benzoic acid;
3-[5-(6-Pyrrolidin-1-yl-pyridin-3-yl)-[1,2,4]oxadiazol-3-yl]-benzoic acid;
3-[5-(6-Morpholin-4-yl-pyridin-3-yl)-[1,2,4]oxadiazol-3-yl]-benzoic acid;
3-[5-(3,4,5,6-Tetrahydro-2H-[1,2′]bipyridinyl-5′-yl)-[1,2,4]oxadiazol-3-yl]-benzoic acid;
3-[5-(2-Fluoro-6-hydroxy-phenyl)-[1,2,4]oxadiazol-3-yl]-benzoic acid;
3-[5-(2-Fluoro-phenyl)-[1,2,4]oxadiazol-3-yl]-benzoic acid methyl ester;
3-[5-(2-Fluoro-phenyl)-[1,2,4]oxadiazol-3-yl]-benzoic acid 2-methoxy-ethyl ester;
3-[5-(2-Fluoro-phenyl)-[1,2,4]oxadiazol-3-yl]-benzoic acid 2-(2-methoxy-ethoxy)-ethyl ester;
3-[5-(2-Fluoro-phenyl)-[1,2,4]oxadiazol-3-yl]-benzoic acid 2-[2-(2-methoxy-ethoxy)-ethoxy]-ethyl ester;
3-[5-(2-Fluoro-phenyl)-[1,2,4]oxadiazol-3-yl]-benzoic acid 2-{2-[2-(2-methoxy-ethoxy)-ethoxy]-ethoxy}-ethyl ester;
3-[5-(2-Fluoro-phenyl)-[1,2,4]oxadiazol-3-yl]-benzoic acid 2-(2-{2-[2-(2-methoxy-ethoxy)-ethoxy]-ethoxy}-ethoxy)-ethyl ester;
3-[5-(2-Fluoro-phenyl)-[1,2,4]oxadiazol-3-yl]-benzoic acid 2-[2-(2-{2-[2-(2-hydroxy-ethoxy)-ethoxy]-ethoxy}-ethoxy)-ethoxy]-ethyl ester;
3-[5-(4-Amino-phenyl)-[1,2,4]oxadiazol-3-yl]-benzoic acid;
3-[5-(4-Azido-phenyl)-[1,2,4]oxadiazol-3-yl]-benzoic acid; and
3-[5-(4-Benzyloxy-phenyl)-[1,2,4]oxadiazol-3-yl]-benzoic acid
   and pharmaceutically acceptable salts, hydrates, solvates and stereoisomers, including enantiomers, diastereomers, racemates or mixtures of stereoisomers, thereof.

In one embodiment, provided herein is a compound having the structure:

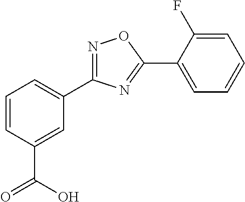 [SMILES: O=C(O)c1cccc(-c2noc(-c3ccccc3F)n2)c1]

and pharmaceutically acceptable salts, hydrates and solvates thereof.

### 4.2 Pharmaceutical Formulations

Pharmaceutical compositions and single unit dosage forms comprising a Compound are also provided herein. Individual dosage forms may be suitable for oral, mucosal (including sublingual, buccal, rectal, nasal, or vaginal) or parenteral (including subcutaneous, intramuscular, bolus injection, intraarterial, or intravenous) administration. In certain embodiments, pharmaceutical compositions and single unit dosage forms are suitable for oral administration.

In one embodiment, the pharmaceutical composition is a solid oral dosage form.

In one embodiment, the pharmaceutical composition is a liquid oral dosage form. In a particular embodiment, present invention provides doses, unit dosage formulations and pharmaceutical compositions wherein a Compound is orally bioavailable. Advantages of oral administration can include ease of administration, higher patient compliance with the dosing regimen, clinical efficacy, fewer complications, shorter hospital stays, and overall cost savings.

In another embodiment, provided herein are unit dosage formulations that comprise between from about 35 mg to about 20 kg, from about 35 mg to about 10 kg, from about 35 mg to about 8 kg, from about 35 mg to about 5 kg, from about 35 mg to about 4 kg, from about 35 mg to about 3 kg, from about 35 mg to about 2 kg, from about 35 mg to about 1500 mg, from about 35 mg to about 1400 mg, from about 125 mg to about 1400 mg, from about 200 mg to about 1400 mg, from about 250 mg to about 1400 mg, from about 300 mg to about 1400 mg, from about 400 mg to about 1400 mg, from about 500 mg to about 1400 mg, from about 600 mg to about 1400 mg, from about 800 mg to about 1400 mg or from about 1000 mg to about 1400 mg of a Compound. In one embodiment, the unit dosage formulation comprises a Compound and one or more carriers or excipients suitable for suspension in a pharmaceutically acceptable solvent or a palatable liquid (e.g., water, milk, juice, fruit sauce, baby food or baby formula).

In another embodiment, provided herein are unit dosage formulations that comprise 35 mg, 50 mg, 70 mg, 100 mg, 125 mg, 140 mg, 150 mg, 175 mg, 200 mg, 250 mg, 280 mg, 300 mg, 350 mg, 400 mg, 450 mg, 500 mg, 560 mg, 600 mg, 700 mg, 750 mg, 800 mg, 900 mg, 1000 mg, 1400 mg, 1500 mg, 2000 mg, 3000 mg, 4000 mg, 5000 mg, 8000 mg, 10000 mg or 20000 mg, of a Compound. In a further embodiment, provided herein are unit dosage formulations that comprise about 35 mg, about 50 mg, about 70 mg, about 100 mg, about 125 mg, about 140 mg, about 150 mg, about 175 mg, about 200 mg, about 250 mg, about 280 mg, about 300 mg, about 350 mg, about 400 mg, about 450 mg, about 500 mg, about 560 mg, about 600 mg, about 700 mg, about 750 mg, about 800 mg, about 900 mg, 1000 mg, 1400 mg, 1500 mg, 2000 mg, 3000 mg, 4000 mg, 5000 mg, 8000 mg, 10000 mg or 20000 mg of a Compound.

In one embodiment, preparing a suspension of a 250 mg unit dosage formulation of a Compound and one or more carriers or excipients provided as a single unit dosage form is carried out by the addition of about 10 mL of a palatable liquid directly in a suitable container containing the single unit dosage form to achieve a concentration of about 25 mg/mL in the total volume of suspension. For a 1000 mg unit dosage formulation of the Compound and one or more carriers or excipients provided as a single unit dosage form, about 20 mL of the palatable liquid is added directly in the container containing the single unit dosage form to achieve a concentration of about 50 mg/mL in the total volume of suspension. Immediately after the palatable liquid is added, the container is closed and shaken gently by hand for at least about 30 seconds to achieve a homogeneous suspension. Although the suspension may remain in the original plastic container for up to 24 hours before ingestion, it is recommended that the suspension be taken shortly after being prepared. If there is a delay of more than about 15 minutes between preparing the suspension and dosing, it is recommended that the container should be reshaken gently by hand for at least about 30 seconds. It is recommended that the suspension be administered directly from the container. If the entire unit dosage form is to be administered, it is further recommended that the container be rinsed once with the palatable liquid and this rinse liquid be ingested to ensure that the entire amount of the single unit dosage form is administered. If a partial amount of the suspension of the single unit dosage form is to be administered, a spoon or syringe can be used to obtain the appropriate dose.

Single unit dosage forms provided herein suitable for oral administration to a patient include, but are not limited to: packets; sachets; cachets; tablets; caplets; capsules, such as soft elastic gelatin capsules; troches; lozenges; dispersions; powders; granules; solutions; liquid dosage forms, including suspensions (e.g., aqueous or non-aqueous liquid suspensions); emulsions (e.g., oil-in-water emulsions, or a water-in-oil liquid emulsion); and elixirs. In one embodiment, the invention relates to a colloid solution or a solution with additional active agent, above the saturating concentration (i.e., suspension). These and other methods of preparing specific dosage forms encompassed by this invention will vary from one another as will be readily apparent to those skilled in the art. See, e.g., Remington's Pharmaceutical Sciences, 18th ed., Mack Publishing, Easton Pa. (1990).

Typical oral dosage forms provided herein are prepared by combining a Compound in an intimate admixture with at least one carrier or excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents (e.g., vanilla extract and the like), preservatives, and coloring agents. Examples of excipients suitable for use in solid oral dosage forms (e.g., powders, granules, tablets, capsules, and caplets) include, but are not limited to, suspending agents, binding agents, surfactant agents, granulating agents and disintegrating agents. In a particular embodiment, examples of excipients suitable for use in solid oral dosage forms (e.g., powders or granules) include polydextrose, polyethylene glycol, poloxamer, mannitol, crospovidone, hydroxyethyl cellulose, flavoring, colloidal silica, and magnesium stearate.

Specific unit dosage formulations include granule or powder formulations comprising an effective amount of a Compound which are suitable for suspending in a pharmaceutically acceptable solvent or a palatable liquid (e.g., water, milk, juice, fruit sauce, baby food or baby formula) and subsequent oral administration. In a particular embodiment, the granule or powder may contain one or more carriers or excipients in combination with the active agent. In another embodiment, the granule or powder can be stored in a sealed container prior to administration or suspension. In yet another embodiment, the granule or powder can be encapsulated (e.g., in a gelatin capsule) or prepared as a tablet dosage form.

In one embodiment, a Compound is provided in unit dose packets or sachets containing white to off-white vanilla-flavored granules. In certain embodiments, each unit dose packet or sachet comprises a Compound with the following inactive ingredients: polydextrose, polyethylene glycol, poloxamer, mannitol, crospovidone, hydroxyethyl cellulose, vanilla flavoring, colloidal silica, and magnesium stearate.

### 4.3 Patient Populations

In some embodiments, a subject treated for MMA in accordance with the methods provided herein is an animal (e.g., a human or non-human animal) that has or is diagnosed with MMA (including mut⁰ MMA, mut⁻ MMA, cblA MMA, and cblB MMA) associated with at least one allele of a gene comprising a mutation (e.g., nonsense mutation) that results in a premature stop codon in RNA encoded by an allele of the gene associated with MMA. In a specific embodiment, a subject treated for MMA in accordance with the methods provided herein is an animal (e.g., a human or non-human animal) that has or is diagnosed with MMA which is associated with at least one allele of the MUT, MMAA (cblA) or MMAB (cblB) gene that contains a mutation (e.g., nonsense mutation) that results in a premature stop codon in RNA encoded by an allele of the MUT, MMAA or MMAB gene. In certain embodiments, a subject treated for MMA in accordance with the methods provided herein is a human who has or is diagnosed with MMA (including mut⁰ MMA, mut⁻ MMA, cblA MMA, and cblB MMA) which is associated with at least one allele of the MUT, MMAA (cblA) or MMAB (cblB) gene that contains a mutation (e.g., nonsense mutation) that results in a premature stop codon in RNA encoded by an allele of the MUT, MMAA or MMAB gene. In specific embodiments, a subject treated for MMA in accordance with the methods provided herein is human that meets one, two or more, or all of the criteria for subjects in the working examples in Section 6 et seq. In another embodiment, a biological sample (e.g., a blood sample) is obtained from a subject with MMA and the MUT, MMAA, and/or MMAB genes are sequenced to determine or confirm the presence of a mutation (e.g., nonsense mutation) that results in a premature stop codon in RNA encoded by the MUT, MMAA, and/or MMAB genes (and in a specific embodiment, the sequencing is conducted prior to administration of a Compound). In one embodiment, the subject is a male human. In another embodiment, the subject is a female human.

In some embodiments, a subject treated for MMA in accordance with the methods provided herein inherited MMA. In certain embodiments, a subject treated for MMA in accordance with the methods provided herein developed MMA as a result of a somatic mutation. In some embodiments, a subject treated for MMA in accordance with the methods provided herein developed MMA spontaneously through gene mutation.

In one embodiment, a subject treated for MMA in accordance with the methods provided herein is a fetus. In accordance with this embodiment, a pregnant female may be administered a Compound in a manner that permits the Compound to pass through the placenta to the fetus. Alternatively, the Compound may be administered directly to the fetus by, e.g., injection.

In one embodiment, a subject treated for MMA in accordance with the methods provided herein is a human infant. In one embodiment, a subject treated for MMA in accordance with the methods provided herein is an elderly human. In another embodiment, a subject treated for MMA in accordance with the methods provided herein is a human adult. In another embodiment, a subject treated for MMA in accordance with the methods provided herein is a human child. In another embodiment, a subject treated for MMA in accordance with the methods provided herein is a human toddler. In a specific embodiment, a subject treated for MMA in accordance with the methods provided herein is a human that is less than 5 years old. In another specific embodiment, a subject treated for MMA in accordance with the methods provided herein is a human that is older than 5 years old. In a specific embodiment, a subject treated for MMA in accordance with the methods provided herein is a human that is less than 5 years old, is older than 5 years old, is 18 years old or is older than 18 years old.

In certain embodiments, a subject treated for MMA in accordance with the methods provided herein is a human that is a newborn, or is about 1 month to 12 months old, about 1 year to 10 years old, about 10 to 20 years old, about 12 to 18 years old, about 20 to 30 years old, about 30 to 40 years old, about 40 to 50 years old, about 50 to 60 years old, about 60 to 70 years old, about 70 to 80 years old, about 80 to 90 years old, about 90 to 100 years old, or any age in between. In a particular embodiment, a subject treated for MMA in accordance with the methods provided herein is a newborn human. In a certain embodiment, a subject treated for MMA in accordance with the methods provided herein is a human that is between the age of newborn and 1 year old. In a certain embodiment, a subject treated for MMA in accordance with the methods provided herein is a human that is between the age of 1 year old and 18 years old. In a certain embodiment, a subject treated for MMA in accordance with the methods provided herein is a human that is between the age of 1 year old and 5 years old. In a certain embodiment, a subject treated for MMA in accordance with the methods provided herein is a human that is between the age of 5 years old or 12 years old. In a certain embodiment, a subject treated for MMA in accordance with the methods provided herein is a human that is between the age of 12 years old and 18 years old. In a certain embodiment, a subject treated for MMA in accordance with the methods provided herein is a human that is at least 1 year old or older. In a certain embodiment, a subject treated for MMA in accordance with the methods provided herein is a human that is at least 2 years old or older. In other embodiments, a subject treated for MMA in accordance with the methods provided herein is a human that is between the ages of 2 years old and 5 years old, 2 years old and 10 years old, 2 years old and 12 years old, 2 years old and 15 years old, 2 years old and 18 years old, 5 years old and 10 years old, 5 years old and 12 years old, 5 years old and 15 years old or 5 years old and 18 years old.

In some embodiments, a subject treated for MMA in accordance with the methods provided herein is administered a Compound or a pharmaceutical composition thereof, or a combination therapy before any adverse effects or intolerance to therapies other than the Compound develops. In some embodiments, a subject treated for MMA in accordance with the methods provided herein is a refractory patient. In a certain embodiment, a refractory patient is an MMA patient that is refractory to a standard therapy (e.g., carnitine or cobalamin supplements).

In some embodiments, a subject treated for MMA in accordance with the methods provided herein is a human that has proven refractory to therapies other than treatment with a Compound, but is no longer on these therapies. In certain embodiments, a subject treated for MMA in accordance with the methods provided herein is a human already receiving one or more conventional MMA therapies, such as camitine supplements, cobalamin supplements, antibiotics, kidney transplant, and/or liver transplant. In specific embodiments, a subject treated for MMA in accordance with the methods provided herein is a human on a low-protein diet. In certain embodiments, a subject treated for MMA in accordance with the methods provided herein is a human on a diet that avoids substances containing isoleucine, threonine, methionine, and valine.

In some embodiments, a subject treated for MMA in accordance with the methods provided herein is a human susceptible to adverse reactions to conventional therapies. In some embodiments, a subject treated for MMA in accordance with the methods provided herein is a human that has not received a therapy, e.g., a carnitine supplement, a cobalamin supplement, an antibiotic, a kidney transplant, and/or a liver transplant, prior to the administration of a Compound or a pharmaceutical composition thereof. In other embodiments, a subject treated for MMA in accordance with the methods provided herein is a human that has received a therapy prior to administration of a Compound or a pharmaceutical composition thereof. In some embodiments, a subject treated for MMA in accordance with the methods provided herein is a human that has experienced adverse side effects to the prior therapy or the prior therapy was discontinued due to unacceptable levels of toxicity to the human.

In specific embodiments, a subject treated for MMA in accordance with the methods provided herein is a human diagnosed with a complete (mut⁰) or partial (mut⁻) defect in methylmalonyl-CoA mutase (MCM). The gene encoding MCM is referred to as the MUT gene and is located on chromosome 6p21.1. In accordance with such embodiments, the subject contains at least one allele of the MUT gene with a mutation (e.g., nonsense mutation) that results in a premature stop codon in RNA encoded by an allele of the MUT gene. Specific examples of mutations (e.g., nonsense mutations) found in the MUT gene include, but are not limited to, those nucleotide changes at one or more of the positions in an exon or intron of the gene, in particular those identified in Table 1, supra that result in a premature stop codon in RNA encoded by an allele of the MUT gene.

In specific embodiments, a subject treated for MMA in accordance with the methods provided herein is a human diagnosed with defects in enzymes required for mitochondrial synthesis of the active form of adenosylcobalamin (AdoCbl), which is a co-factor for MCM. Different complementation groups, namely cblA and cblB have been identified in which the synthesis of AdoCbl is blocked. The MMAA and MMAB genes responsible for the cblA and cblB disorders associated with MMA are located on chromosomes 4 and 12, respectively. In accordance with such embodiments, the subject contains at least one allele of the MMAA or MMAB gene with a mutation (e.g., nonsense mutation) that results in a premature stop codon in RNA encoded by an allele of the MMAA or MMAB gene. Specific examples of mutations (e.g., nonsense mutations) found in the MMAA gene include, but are not limited to, to those nucleotide changes at one or more of the positions in an exon or intron of the gene, in particular those identified in Table 2, supra that result in a premature stop codon in RNA encoded by the MMAA gene. Specific examples of mutations (e.g., nonsense mutations) found in the MMAB gene include, but are not limited to, those intronic nucleotide changes at one or more of the positions in an exon or intron of the gene, in particular those identified in Table 3, supra that result in a premature stop codon in RNA encoded by an allele of the MMAB gene.

In certain embodiments, a subject treated for MMA in accordance with the methods provided herein is a human diagnosed with MMA whose cultured cells (e.g., fibroblasts or lymphocytes) are responsive to a Compound or a pharmaceutical composition thereof. In other words, subjects treated for MMA include those that have cells which when cultured in vitro in the presence of a Compound or a pharmaceutical composition thereof have an increase in cellular enzyme activity (e.g., an increase in MCM activity or an increase in the activity of an enzyme required for the active form of AdoCbl) relative to the cellular enzyme activity in the absence of the Compound or pharmaceutical composition thereof, as determined by techniques known to one skilled in the art or as described herein (see, e.g., Section 6 et. seq., infra). In a specific embodiment, the responsiveness of a patient's cells to a Compound is assessed before the patient receives a Compound or a pharmaceutical composition thereof.

In some embodiments, a subject treated for MMA in accordance with the methods provided herein is a human diagnosed with MMA experiencing one, two or more of the following symptoms: metabolic acidosis, lethargy, vomiting, dehydration, hypotonia, hypoglycemia, repeated yeast infections, renal impairment, mental retardation, developmental delays, seizures, movement disorders, progressive encephalopathy, facial dysmorphism (e.g., high forehead, broad nasal bridge, epicanthal folds, long smooth philtrum, or triangular mouth), skin lesions (e.g., moniliasis), occasional hepatomegaly, acute onset of choreoathetosis, dystonia, dysphagia, or dysarthria, and reduced glomerular filtration rate (GFR).

In some embodiments, a subject treated for MMA in accordance with the methods provided herein is not, has not and/or will not receive a drug that is primarily metabolized by CYP2C9. In particular embodiments, a subject treated for MMA in accordance with the methods provided herein has not and will not received a drug that is primarily metabolized by CYP2C9 1, 2, 3 or 4 weeks before receiving a Compound or a pharmaceutical composition thereof and 1, 2, 3 or 4 weeks after receiving the Compound or pharmaceutical composition. Examples of such drugs include, without limitation, warfarin and phenylonin.

In some embodiments, a subject treated for MMA in accordance with the methods provided herein does not have a hypersensitivity to one or more of the following: polydextrose, polyethylene glycol, poloxamer, mannitol, crospovidone, hydroxyethyl cellulose, vanilla, colloidal silica, or magnesium stearate. In certain embodiments, a subject treated for MMA in accordance with the methods provided herein is not or has not undergoing dialysis for renal dysfunction. In certain embodiments, a subject treated for MMA in accordance with the methods provided herein has not or will not have an organ transplant. In some embodiments, a subject treated for MMA in accordance with the methods provided herein has not experienced any change in prescribed diet, protein intake of caloric intake within 2 weeks, 1 month, 2 months, 3 months, 4 months or more of initiation of administration of a Compound. In certain embodiments, a subject treated for MMA in accordance with the methods provided herein has not experienced an episode of metabolic decompensation within 1 week, 2 weeks, 3 weeks, 1 month, 2 months, 3 months, 4 months or more of initiation of administration of a Compound. In some embodiments, a subject treated for MMA in accordance with the methods provided herein has not experienced a change in the dosage of a carnitine supplement, a cobalamin supplement, or an antibiotic for gut flora.

### 4.4 Dosage and Administration

In accordance with the methods for treating MMA provided herein, a Compound or a pharmaceutical composition thereof can be administered to a subject in need thereof by a variety of routes in amounts which result in a beneficial or therapeutic effect. A Compound or pharmaceutical composition thereof may be orally administered to a subject in need thereof in accordance with the methods for treating MMA provided herein. The oral administration of a Compound or a pharmaceutical composition thereof may facilitate subjects in need of such treatment complying with a regimen for taking the Compound or pharmaceutical composition. Thus, in a specific embodiment, a compound or pharmaceutical composition thereof is administered orally to a subject in thereof.

Other routes of administration include, but are not limited to, intravenous, intrathecal, intradermal, intramuscular, subcutaneous, intranasal, inhalation, transdermal, topical, transmucosal, intracranial, intratumoral, epidural and intra-synovial. In one embodiment, a Compound or a pharmaceutical composition thereof is administered systemically (e.g., parenterally) to a subject in need thereof. In another embodiment, a Compound or a pharmaceutical composition thereof is administered locally to a subject in need thereof. In one embodiment, a Compound or a pharmaceutical composition thereof is administered intrathecally or via a route that permits the Compound to cross the blood-brain barrier (e.g., orally).

In accordance with the methods for treating MMA provided herein that involve administration of a Compound in combination with one or more additional therapies, the Compound and one or more additional therapies may be administered by the same route or a different route of administration.

The dosage and frequency of administration of a Compound or a pharmaceutical composition thereof is administered to a subject in need thereof in accordance with the methods for treating MMA provided herein will be efficacious while minimizing any side effects. The exact dosage and frequency of administration of a Compound or a pharmaceutical composition thereof can be determined by a practitioner, in light of factors related to the subject that requires treatment. Factors which may be taken into account include the severity of the disease state, general health of the subject, age, weight, and gender of the subject, diet, time and frequency of administration, drug combination(s), reaction sensitivities, and tolerance/response to therapy. The dosage and frequency of administration of a Compound or a pharmaceutical composition thereof may be adjusted over time to provide sufficient levels of the Compound or to maintain the desired effect.

In certain embodiments, a Compound or a pharmaceutical composition thereof is administered to a subject in need thereof in accordance with the methods for treating MMA provided herein at a dosage and a frequency of administration that achieves one or more of the following: (i) the reduction or amelioration of the severity of one or more MMA symptoms; (ii) the reduction in the duration of one or more symptoms associated with MMA; (iii) the prevention in the recurrence of a symptom associated with MMA; (iv) the reduction in hospitalization of a subject; (v) a reduction in hospitalization length; (vi) the increase in the survival of a subject; (vii) the enhancement or improvement of the therapeutic effect of another therapy; (viii) an improvement in developmental or cognitive ability; (ix) a decrease in the frequency and/or number of metabolic decompensation episodes; (x) an improvement in control of muscle contraction; (xi) a reduction in mortality; (xii) an increase in the survival rate of patients; (xiii) a decrease in hospitalization rate; (xiv) the prevention of the development or onset of one or more symptoms associated with MMA; (xv) the reduction in the number of symptoms associated with MMA; (xvi) an decrease in the concentration of MMacid in biological fluids (e.g., plasma or urine); (xvii) a decrease in the concentration of metabolites of MMacid, such as propionylcarnitine or methylcitrate, in biological fluids (e.g., plasma or urine); (xviii) a decrease in erythrocyte OLCFA levels; (xix) an increase in the urinary urea:MMacid ratio; (xx) an increase in symptom-free survival of MMA patients; (xxi) an improvement in renal function; and (xxii) improvement in quality of life as assessed by methods well known in the art.

In one embodiment, a Compound or a pharmaceutical composition thereof is administered to a subject in need thereof in accordance with the methods for treating MMA provided herein once in a 6, 12 or 24 hour period, wherein each administration can be separated by about 4-14 hours. In another embodiment, a Compound or a pharmaceutical composition thereof is administered to a subject in need thereof in accordance with the methods for treating MMA provided herein two times in a 12 or 24 hour period, wherein each administration can be separated by about 4-14 hours. In another embodiment, a Compound or a pharmaceutical composition thereof is administered to a subject in need thereof in accordance with the methods for treating MMA provided herein two times in a 12 or 24 hour period, wherein each administration can be separated by about 4-8 hours. In another embodiment, a Compound or a pharmaceutical composition thereof is administered to a subject in need thereof in accordance with the methods for treating MMA provided herein two times in a 12 or 24 hour period, wherein each administration can be separated by about 6 hours. In another embodiment, a Compound or a pharmaceutical composition thereof is administered to a subject in need thereof in accordance with the methods for treating MMA provided herein three times in a 24 hour period, wherein a first dose, a second dose and a third dose can be separated by about 4-14 hours. In another embodiment, a Compound or a pharmaceutical composition thereof is administered to a subject in need thereof in accordance with the methods for treating MMA provided herein three times in a 24 hour period, wherein a first dose, a second dose and a third dose can be separated by about 4-8 hours. In another embodiment, a Compound or a pharmaceutical composition thereof is administered to a subject in need thereof in accordance with the methods for treating MMA provided herein three times in a 24 hour period, wherein a first dose, a second dose and a third dose can be separated by about 6 hours. In another embodiment, a Compound or a pharmaceutical composition thereof is administered to a subject in need thereof in accordance with the methods for treating MMA provided herein three times in a subsequent 24 hour period, wherein a first dose for the subsequent 24 hour period can be separated by about 8-16 hours from the third dose administered in a previous 24 hour period. In another embodiment, a Compound or a pharmaceutical composition thereof is administered to a subject in need thereof in accordance with the methods for treating MMA provided herein three times in a subsequent 24 hour period, wherein a first dose for the subsequent 24 hour period can be separated by about 12 hours from the third dose administered in a previous 24 hour period. In these embodiments, the Compound or a pharmaceutical composition thereof can be administered, for example, at meal time, such as breakfast and supper. In these embodiments, the first dose, the second dose and the third dose of the Compound or a pharmaceutical composition thereof can be administered about 30 minutes after a meal. In certain embodiments, a suspension (such as in water, milk or juice) of a single unit dosage form is administered orally three times every day in three separate doses. In further embodiments, the amounts of the first dose and the second dose are the same and the third dose is twice the amount of the first dose. Doses can be taken in the morning (first dose), at midday (second dose), and in the evening (third dose). Approximate intervals for dosing include 6 hours between morning and midday doses, 6 hours between midday and evening doses, and 12 hours between the evening dose and the morning dose on the next day. In a particular embodiment, a Compound or a pharmaceutical composition thereof is administered once in the morning, once in the afternoon and once in the evening. Intervals between doses include a period of about 3, 4, 5, 6, 7, 8, 9, 10, 11, 12, 13, 14 or 16 hours.

In one embodiment, the dose of a Compound is titrated throughout a 24 hour period. In another embodiment, the second dose administered is escalated (e.g., doubled). In another embodiment, the first and second dose administered are kept constant and the third dose administered is escalated (e.g., doubled). In a particular embodiment, the three doses in a 24 hour period are administered according to the formula: 1×, 1×, 2×, where X is a particular initial dose (e.g., 4 mg/kg, 5 mg/kg, 6 mg/kg, 7 mg/kg, 8 mg/kg, 10 mg/kg, 12 mg/kg, 14 mg/kg, 16 mg/kg, 18 mg/kg, 20 mg/kg, 22 mg/kg, 24 mg/kg, 25 mg/kg, 26 mg/kg, 28 mg/kg, 30 mg/kg, 32 mg/kg, 34 mg/kg, 35 mg/kg, 36 mg/kg, 38 mg/kg, 40 mg/kg, 42 mg/kg, 44 mg/kg, 45 mg/kg, 46 mg/kg, 48 mg/kg or 50 mg/kg). In another embodiment, the Compound or a pharmaceutical composition thereof is administered within (i.e., before or after) about 10, 15, 30, 45 or 60 minutes of the patient having food. In another embodiment, the Compound or a pharmaceutical composition thereof is administered within about 30 minutes of the patient having a meal. In one embodiment, an effective amount of a Compound or a pharmaceutical composition thereof is sprinkled on or mixed in food. In another embodiment, a Compound or a pharmaceutical composition thereof is administered without food.

An illustrative dosing regimen is that wherein a patient is administered a Compound or a pharmaceutical composition thereof within 30 minutes after a meal at approximately 6-hour intervals (e.g., at about 7:00 AM after breakfast, about 1:00 PM after lunch, and at about 7:00 PM after supper).

In yet another embodiment, a Compound or a pharmaceutical composition thereof is administered to a subject in need thereof in accordance with the methods for treating MMA provided herein in single or divided (e.g., three times in a 24 hour period) doses, wherein the amount for each of the three doses is determined by patient weight. According to a weight-based dosing regimen, each dose administered may be in a range of between 0.1 mg/kg and 500 mg/kg, 1 mg/kg and 250 mg/kg, 1 mg/kg and 150 mg/kg, 1 mg/kg and 100 mg/kg, 1 mg/kg and 50 mg/kg, 1 mg/kg and 48 mg/kg, 1 mg/kg and 46 mg/kg, 1 mg/kg and 45 mg/kg, 1 mg/kg and 44 mg/kg, 1 mg/kg and 42 mg/kg, 1 mg/kg and 40 mg/kg, 1 mg/kg and 38 mg/kg, 1 mg/kg and 36 mg/kg, 1 mg/kg and 35 mg/kg, 1 mg/kg and 34 mg/kg, 1 mg/kg and 32 mg/kg, 1 mg/kg and 30 mg/kg, 1 mg/kg and 28 mg/kg, 1 mg/kg and 26 mg/kg, 1 mg/kg and 25 mg/kg, 1 mg/kg and 24 mg/kg, 1 mg/kg and 22 mg/kg, 1 mg/kg and 20 mg/kg, 1 mg/kg and 18 mg/kg, 1 mg/kg and 16 mg/kg, 1 mg/kg and 14 mg/kg, 1 mg/kg and 12 mg/kg, 1 mg/kg and 10 mg/kg, 1 mg/kg and 8 mg/kg, 1 mg/kg and 7 mg/kg, 1 mg/kg and 6 mg/kg, 1 mg/kg and 5 mg/kg, 1 mg/kg and 4 mg/kg, or 2 mg/kg and 10 mg/kg to a patient in need thereof.

In a particular embodiment, a Compound or a pharmaceutical composition thereof is administered in a dose of about 2-6 mg/kg, about 3-7 mg/kg, about 5-9 mg/kg, about 6-10 mg/kg, about 8-12 mg/kg, about 10-14 mg/kg, about 12-16 mg/kg, about 14-18 mg/kg, about 16-20 mg/kg, about 18-22 mg/kg, about 20-24 mg/kg, about 22-26 mg/kg, about 24-28 mg/kg, about 26-30 mg/kg, about 28-32 mg/kg, about 30-34 mg/kg, about 32-36 mg/kg, about 34-38 mg/kg, about 36-40 mg/kg, about 38-42 mg/kg, about 40-44 mg/kg, about 42-46 mg/kg, about 44-48 mg/kg, about 46-50 mg/kg, or about 48-52 mg/kg. In a particular embodiment, a Compound or a pharmaceutical composition thereof is administered in a dose of about 4 mg/kg, about 5 mg/kg, about 6 mg/kg, about 7 mg/kg, about 8 mg/kg, about 10 mg/kg, about 12 mg/kg, about 14 mg/kg, 16 mg/kg, 18 mg/kg, about 20 mg/kg, 22 mg/kg, 24 mg/kg, about 25 mg/kg, 26 mg/kg, 28 mg/kg, about 30 mg/kg, 32 mg/kg, 34 mg/kg, about 35 mg/kg, 36 mg/kg, 38 mg/kg, about 40 mg/kg, 42 mg/kg, 44 mg/kg, 45 mg/kg, 46 mg/kg, 48 mg/kg or about 50 mg/kg. In another embodiment, any dose of a Compound or a pharmaceutical composition thereof described in the preceding embodiment is administered three times in a 24 hour period.

In another embodiment, provide herein are methods for continuous therapy wherein a Compound or a pharmaceutical composition thereof is administered to a subject in need thereof in accordance with the methods for treating MMA provided herein daily for a certain period of time (e.g., 5, 7, 10, 14, 20, 24, 28, 60 or 120 days or more). In one embodiment, a Compound or a pharmaceutical composition thereof is continuously administered three times per 24 hour period. In another embodiment, a Compound or a pharmaceutical composition thereof is administered continuously in subsequent 24 hour periods daily, weekly, monthly or yearly. In a specific embodiment, a Compound or a pharmaceutical composition thereof is continuously administered three times per 24 hour period and subsequent 24 hour periods at doses of about 4 mg/kg, about 4 mg/kg and about 8 mg/kg for days, weeks, months or years. In a specific embodiment, a Compound or a pharmaceutical composition thereof is continuously administered three times per 24 hour period and subsequent 24 hour periods at doses of about 5 mg/kg, about 5 mg/kg and about 10 mg/kg for days, weeks, months or years. In a specific embodiment, a Compound or a pharmaceutical composition thereof is continuously administered three times per 24 hour period and subsequent 24 hour periods at doses of about 7 mg/kg, about 7 mg/kg and about 14 mg/kg for days, weeks, months or years. In a specific embodiment, a Compound or a pharmaceutical composition thereof is continuously administered three times per 24 hour period and subsequent 24 hour periods at doses of about 10 mg/kg, about 10 mg/kg and about 20 mg/kg for days, weeks, months or years. In a specific embodiment, a Compound or a pharmaceutical composition thereof is continuously administered three times per 24 hour period and subsequent 24 hour periods at doses of about 20 mg/kg, about 20 mg/kg and about 40 mg/kg for days, weeks, months or years. In a specific embodiment, a Compound or a pharmaceutical composition thereof is continuously administered three times per 24 hour period and subsequent 24 hour periods at doses of 10 mg/kg, 10 mg/kg and 20 mg/kg for days, weeks, months or years. In a specific embodiment, a Compound or a pharmaceutical composition thereof is continuously administered three times per 24 hour period and subsequent 24 hour periods at doses of 20 mg/kg, about 20 mg/kg and 40 mg/kg for days, weeks, months or years.

Treatment periods for a course of therapy can span one week, two weeks, three weeks, four weeks, five weeks, six weeks, seven weeks, eight weeks, nine weeks, ten weeks, eleven weeks, twelve weeks, thirteen weeks, fourteen weeks, four months, five months, six months, seven months, eight months, nine months, ten months, eleven months, one year, two years, three years, four years, five years or longer. The treatment periods can be interrupted by periods of rest which can span a day, one week, two weeks, three weeks, four weeks, five weeks, six weeks, seven weeks, eight weeks, nine weeks, ten weeks, eleven weeks, twelve weeks, thirteen weeks, fourteen weeks, four months, five months, six months, seven months, eight months, nine months, ten months, eleven months, one year, two years, three years, four years, five years or longer. Such determinations can be made by one skilled in the art (e.g., a physician).

In a particular embodiment, initial treatment is continuous for 28 days, followed by no treatment for 21-42 days, followed by continuous treatment for an additional 28 days, followed by no treatment for 14 days, optionally followed by an additional round of treatment. In one embodiment, the dose given during the second 28 days of treatment is greater than that given during the first 28 days of initial treatment. One such embodiment includes an example, wherein a patient in need thereof is administered three doses of a Compound or a pharmaceutical composition thereof (e.g., 5 mg/kg, 5 mg/kg and 10 mg/kg) in a 24 hour period (for example, in the morning, at midday and in the evening) for 28 continuous days of initial treatment, followed by 21-42 days without treatment, followed by administration of three doses of a Compound or a pharmaceutical composition thereof (e.g., 20 mg/kg, 20 mg/kg and 40 mg/kg) in a 24 hour period (for example, in the morning, at midday and in the evening) for an additional 28 continuous days. Another such embodiment includes an example, wherein a patient in need thereof is administered three doses of a Compound or a pharmaceutical composition thereof (e.g., 5 mg/kg, 5 mg/kg and 10 mg/kg) in a 24 hour period (for example, in the morning, at midday and in the evening) for 28 continuous days of initial treatment, followed by 21-42 days without treatment, followed by administration of three doses of a Compound or a pharmaceutical composition thereof (e.g., 10 mg/kg, 10 mg/kg and 20 mg/kg) in a 24 hour period (for example, in the morning, at midday and in the evening) for an additional 28 continuous days.

In another embodiment, the initial round of treatment is followed by no treatment for 14 days, followed by a second round of treatment wherein a patient in need thereof is administered three doses of a Compound or a pharmaceutical composition thereof (e.g., 5 mg/kg, 5 mg/kg and 10 mg/kg) in a 24 hour period (for example, in the morning, at midday and in the evening) for 28 continuous days, followed by 21-42 days without treatment, followed by administration of three doses of a Compound or a pharmaceutical composition thereof (e.g., 20 mg/kg, 20 mg/kg and 40 mg/kg) in a 24 hour period (for example, in the morning, at midday and in the evening) for a third round of treatment for 28 continuous days. In another embodiment, the initial round of treatment is followed by no treatment for 14 days, followed by a second round of treatment wherein a patient in need thereof is administered three doses of a Compound or a pharmaceutical composition thereof (e.g., 5 mg/kg, 5 mg/kg and 10 mg/kg) in a 24 hour period (for example, in the morning, at midday and in the evening) for 28 continuous days, followed by 21-42 days without treatment, followed by administration of three doses of a Compound or a pharmaceutical composition thereof (e.g., 10 mg/kg, 10 mg/kg and 20 mg/kg) in a 24 hour period (for example, in the morning, at midday and in the evening) for a third round of treatment for 28 continuous days.

In a particular embodiment, treatment is continuous for 14 days, followed by no treatment for 14 days, followed by continuous treatment for an additional 14 days. In one embodiment, the dose given during the second 14 days of treatment is greater than that given during the first 14 days of treatment. As a non-limiting example, a patient in need thereof is administered three doses of a Compound or a pharmaceutical composition thereof (e.g., 4 mg/kg, 4 mg/kg and 8 mg/kg) in a 24 hour period for 14 continuous days, followed by 14 days without treatment, followed by administration of three doses of a Compound or a pharmaceutical composition thereof (e.g., 10 mg/kg, 10 mg/kg and 20 mg/kg) in a 24 hour period for an additional 14 continuous days.

In another embodiment, treatment is continuous for 28 days. Continuous treatment can be interrupted by one or more days, months, weeks or years. Continuous treatment can also be followed by a rest period lasting one or more days, months, weeks or years, with continuous treatment then resuming after the rest period.

In some embodiments, a method for treating MMA presented herein involves the administration to a subject in need thereof of one or more doses of an effective amount of a Compound or a pharmaceutical composition, wherein the effective amount may or may not be the same for each dose. In particular embodiments, a first dose of a Compound or pharmaceutical composition thereof is administered to a subject in need thereof for a first period of time, and subsequently, a second dose of a Compound is administered to the subject for a second period of time. The first dose may be more than the second dose, or the first dose may be less than the second dose. A third dose of a Compound also may be administered to a subject in need thereof for a third period of time.

The length of time that a subject in need thereof is administered a Compound or a pharmaceutical composition thereof in accordance with the methods for treating MMA presented herein will be the time period that is determined to be efficacious. In certain embodiments, a method for treating MMA presented herein involves the administration of a Compound or a pharmaceutical composition thereof for a period of time until the severity and/or number of symptoms associated with MMA decrease. In some embodiments, a method for treating MMA presented herein involves the administration of a Compound or a pharmaceutical composition thereof for up to 56 days. In other embodiments, a method for treating MMA presented herein involves the administration of a Compound or a pharmaceutical composition thereof for up to about 4 weeks, 8 weeks, 12 weeks, 16 week, 20 weeks, 24 weeks, 26 weeks (0.5 year), 52 weeks (1 year), 78 weeks (1.5 years), 104 weeks (2 years), or 130 weeks (2.5 years) or more. In certain embodiments, a method for treating MMA presented herein involves the administration of a Compound or a pharmaceutical composition thereof for an indefinite period of time. In some embodiments, a method for treating MMA presented herein involves the administration of a Compound or a pharmaceutical composition thereof for a period of time followed by a period of rest (i.e., a period wherein the Compound is not administered) before the administration of the Compound or pharmaceutical composition thereof is resumed. In specific embodiments, a method for treating MMA presented herein involves the administration of a Compound or a pharmaceutical composition thereof in cycles, e.g., 1 week cycles, 2 week cycles, 3 week cycles, 4 week cycles, 5 week cycles, 6 week cycles, 8 week cycles, 9 week cycles, 10 week cycles, 11 week cycles, or 12 week cycles. In particular embodiments, a method for treating a MMA presented herein involves the administration of a Compound or a pharmaceutical composition thereof three times daily in 4 week cycles.

It will be understood that the amounts of a Compound or a pharmaceutical composition thereof administered to a patient in need thereof are or can be calculated based upon the actual weight of the patient in question or the average weight of the patient population in question.

### 4.5 Combination Therapy

Presented herein are combination therapies for the treatment of MMA which involve the administration of a Compound in combination with one or more additional therapies to a subject in need thereof. In a specific embodiment, presented herein are combination therapies for the treatment of MMA which involve the administration of an effective amount of a Compound in combination with an effective amount of another therapy to a subject in need thereof. Specific examples of such other therapies include, but are not limited to, carnitine supplements (such as L-carnitine), cobalamin supplements and antibiotics (such as metronidazole).

As used herein, the term “in combination,” refers, in the context of the administration of a Compound, to the administration of a Compound prior to, concurrently with, or subsequent to the administration of one or more additional therapies (e.g., agents, surgery, or radiation) for use in treating MMA. The use of the term “in combination” does not restrict the order in which one or more Compounds and one or more additional therapies are administered to a subject. In specific embodiments, the interval of time between the administration of a Compound and the administration of one or more additional therapies may be about 1-5 minutes, 1-30 minutes, 30 minutes to 60 minutes, 1 hour, 1-2 hours, 2-6 hours, 2-12 hours, 12-24 hours, 1-2 days, 2 days, 3 days, 4 days, 5 days, 6 days, 7 days, 1 week, 2 weeks, 3 weeks, 4 weeks, 5 weeks, 6 weeks, 7 weeks, 8 weeks, 9 weeks, 10 weeks, 15 weeks, 20 weeks, 26 weeks, 52 weeks, 11-15 weeks, 15-20 weeks, 20-30 weeks, 30-40 weeks, 40-50 weeks, 1 month, 2 months, 3 months, 4 months 5 months, 6 months, 7 months, 8 months, 9 months, 10 months, 11 months, 12 months, 1 year, 2 years, or any period of time in between. In certain embodiments, a Compound and one or more additional therapies are administered less than 1 day, 1 week, 2 weeks, 3 weeks, 4 weeks, one month, 2 months, 3 months, 6 months, 1 year, 2 years, or 5 years apart.

In some embodiments, the combination therapies provided herein involve administering a Compound daily, and administering one or more additional therapies once a week, once every 2 weeks, once every 3 weeks, once every 4 weeks, once every month, once every 2 months (e.g., approximately 8 weeks), once every 3 months (e.g., approximately 12 weeks), or once every 4 months (e.g., approximately 16 weeks). In certain embodiments, a Compound and one or more additional therapies are cyclically administered to a subject. Cycling therapy involves the administration of the Compound for a period of time, followed by the administration of one or more additional therapies for a period of time, and repeating this sequential administration. In certain embodiments, cycling therapy may also include a period of rest where the Compound or the additional therapy is not administered for a period of time (e.g., 2 days, 3 days, 4 days, 5 days, 6 days, 7 days, 1 week, 2 weeks, 3 weeks, 4 weeks, 5 weeks, 10 weeks, 20 weeks, 1 month, 2 months, 3 months, 4 months, 5 months, 6 months, 7 months, 8 months, 9 months, 10 months, 11 months, 12 months, 2 years, or 3 years). In an embodiment, the number of cycles administered is from 1 to 12 cycles, from 2 to 10 cycles, or from 2 to 8 cycles.

In some embodiments, the methods for treating MMA provided herein comprise administering a Compound as a single agent for a period of time prior to administering the Compound in combination with an additional therapy. In certain embodiments, the methods for treating MMA provided herein comprise administering an additional therapy alone for a period of time prior to administering a Compound in combination with the additional therapy.

In some embodiments, the administration of a Compound and one or more additional therapies in accordance with the methods presented herein have an additive effect relative the administration of the Compound or said one or more additional therapies alone. In some embodiments, the administration of a Compound and one or more additional therapies in accordance with the methods presented herein have a synergistic effect relative to the administration of the Compound or said one or more additional therapies alone.

As used herein, the term “synergistic,” refers to the effect of the administration of a Compound in combination with one or more additional therapies (e.g., agents), which combination is more effective than the additive effects of any two or more single therapies (e.g., agents). In a specific embodiment, a synergistic effect of a combination therapy permits the use of lower dosages (e.g., sub-optimal doses) of a Compound or an additional therapy and/or less frequent administration of a Compound or an additional therapy to a subject. In certain embodiments, the ability to utilize lower dosages of a Compound or an additional therapy and/or to administer a Compound or said additional therapy less frequently reduces the toxicity associated with the administration of a Compound or said additional therapy, respectively, to a subject without reducing the efficacy of a Compound or said additional therapy, respectively, in the treatment of MMA. In some embodiments, a synergistic effect results in improved efficacy of a Compound and each of said additional therapies in treating MMA. In some embodiments, a synergistic effect of a combination of a Compound and one or more additional therapies avoids or reduces adverse or unwanted side effects associated with the use of any single therapy.

The combination of a Compound and one or more additional therapies can be administered to a subject in the same pharmaceutical composition. Alternatively, a Compound and one or more additional therapies can be administered concurrently to a subject in separate pharmaceutical compositions. A Compound and one or more additional therapies can be administered sequentially to a subject in separate pharmaceutical compositions. A Compound and one or more additional therapies may also be administered to a subject by the same or different routes of administration.

The combination therapies provided herein involve administrating to a subject to in need thereof a Compound or a pharmaceutical composition thereof in combination with conventional, or known, therapies for MMA. Current therapies for MMA, include camitine supplements, cobalamin supplements and antibiotics. Other therapies for MMA or a condition associated therewith are aimed at controlling or relieving symptoms, e.g., anti-seizure medication. Accordingly, in some embodiments, the combination therapies provided herein involve administrating to a subject to in need thereof a pain reliever, a medication for epileptic seizures, or other therapy aimed at alleviating or controlling symptoms associated with MMA or a condition associated therewith.

In specific embodiments, the combination therapies provided herein involve administering to a subject in need thereof a Compound or a pharmaceutical composition thereof in combination with one or more of the following: a camitine supplement (e.g., L-camitine), a cobalamin supplement and an antibiotic. In certain embodiments, the combination therapies provided herein involve administering to a subject in need thereof a Compound or a pharmaceutical composition thereof in combination with an organ transplant (e.g., a kidney, liver or kidney and liver transplant).

### 5. EXAMPLE

### Preparation of Compounds Provided Herein

The following examples are presented by way of illustration not limitation.

Compounds provided herein can be prepared by those skilled in the art, such as by the synthetic methods set forth in U.S. Pat. No. 6,992,096 B2, issued Jan. 31, 2006, and U.S. Pat. No. 7,678,922 B2, issued Mar. 16, 2010, both of which are incorporated by reference in their entirety.

### 6. EXAMPLE

### Protocol for Treating Patients

Subjects with MMA may receive doses of a Compound in two cycles with each cycle comprising 28 days of continuous daily administration of a Compound followed by a period of days without administration of the Compound. The first 28 day cycle of daily administration of a Compound may comprise the administration of 5 mg/kg of the Compound in the morning (e.g., 7:00 am+/−1 hour), 5 mg/kg of the Compound midday (e.g., 1:00 pm+/−1 hour), and 10 mg/kg of the Compound in the evening (e.g., 7:00 pm+/−1 hour). After the first cycle of administration of the Compound, subjects may not receive the Compound for a period of 21 to 42 days, or longer as appropriate. The second 28 day cycle of daily administration of the Compound may comprise administration of 20 mg/kg of the Compound in the morning (e.g., 7:00 am+/−1 hour), 20 mg/kg of the Compound midday (e.g., 1:00 pm+/−1 hour), and 40 mg/kg of the Compound in the evening (e.g., 7:00 pm+/−1 hour). After the second cycle of administration of the Compound, subjects may not receive the Compound for a period of 14 days or longer as appropriate. Each cycle of administration of the Compound may be repeated two or more times as appropriate. In a specific embodiment, the Compound is 3-[5-(2-fluoro-phenyl)[1, 2, 4]oxadiazol-3-byl]-benzoic, also known as TRANSLARNA™ (brand of ataluren).

### Clinical Objectives

Efficacy of a Compound for treating MMA may be assessed by determining the effects of the Compound on reduction of plasma methylmalonic acid (MMacid), The efficacy of a Compound for treating MMA may also be assessed by: (i) determining the effect on urinary levels of methylcitrate; (ii) determining the effect on plasma levels of propionlycarnitine; (iii) evaluating effects on erythrocyte odd long-chain fatty acid levels; (iv) determining effects on the urinary urea:MMacid ratio; (v) determining the effects on enzyme activity in cultured fibroblasts and lymphocytes from subjects with MMA, (vi) evaluating the effects on the developmental and cognitive ability of subjects; (vii) evaluating the effects on the dystonia rating scale; (viii) evaluating the effects on the occurrence of any metabolic decompensation episodes; (ix) evaluating the safety profile of the Compound; (x) evaluating compliance with treatment with the Compound; and (xi) determining the Compound's plasma exposure over time.

### Clinical Endpoints

A primary clinical endpoint for efficacy of a Compound for treating MMA includes a reduction in plasma MMacid levels. Other clinical endpoints for the efficacy of a Compound for treating MMA may include:
a reduction in urinary MMacid levels;
a reduction in urinary methylcitrate;
a reduction in plasma propionylcarnitine;
a reduction in erythrocyte odd long-chain fatty acid levels;
an increase in the urea:MMacid ratio;
an improvement in cognitive ability;
a decrease in abnormal tonicity of muscle;
a decrease in hospitalization;
a reduction or no laboratory abnormalities
a decrease in occurrences of episodes of metabolic decompensation;
overall safety profile of a Compound characterized in terms of the type, frequency,
severity, timing, and relationship to the therapy of any adverse events or abnormalities of physical findings, laboratory tests, or ECGs; treatment discontinuations due to adverse events; or serious adverse events;
pharmacokinetic parameters, e.g., time to maximum plasma concentration (Tₘₐₓ), Cₘₐₓ, AUC, terminal elimination half-life (t_{1/2}) based on a Compound's plasma concentrations as assessed by a validated bioanalytical method; and

### Evaluation of Clinical Endpoints

Plasma MMacid Levels:

Plasma MMacid levels may be used to indicate the effectiveness of a Compound to increase the activity of the relevant enzyme or factor {e.g., MCM, cblA, or cblB). Normal plasma MMacid level is <0.27 μmol/L (Fowler et al., 2008, J. Inherit. Metab. Dis. 31: 350-360). Plasma MMacid levels are elevated in patients with MMA, generally in the range of 100 to 1.000 μmol/L in cobalamin-non responsive patients and 5 to 100 μmol/L in cobalamin-responsive patients (Venditti, 2007, Gene Reviews). MMacid is considered to be nephrotoxic, and central nervous system trapping of MMacid, propionyl-CoA, and 2 methylcitrate is considered to be the basis for chronic neurologic complications of MMA (Morath et al., 2008, J. Inherit. Metab. Dis. 31: 35-43). A decrease by >30% in plasma or urine MMacid levels has been considered by some to be a clinically relevant difference (Zwickler et al., 2008, J. Inherit. Metab. Dis. 31: 361-367).

Blood may be collected and plasma MMacid concentrations may be determined using a standard gas chromatography/mass spectroscopy (GC/MS) stable-isotope dilution method.

Urinary MMacid Levels:

Urinary MMacid levels may be used to indicate the effectiveness of a Compound to increase the activity of the relevant enzyme or factor (e.g., MCM, cblA, or cblB). The normal urinary MMacid level is <4 mmol/mol creatinine (Venditti, 2007, Gene Reviews), and the level is significantly elevated in MMA. In general, the more severe types of MMA, mut⁰ and cblB, have higher urinary MMacid levels (about 5,000 to >10.000 mmol/mol creatinine) compared to the less severe types, mut⁻ and cblA (<1,000 to >5,000 mol/mol creatinine) (Horster et al., 2007, Pediatr. Res. 62: 225-230; Fowler et al., 2008, J. Inherit. Metab. Dis. 31: 350-360). It has been observed that chronic renal failure does not occur in patients with urinary MMacid levels below −2.000 mmol/mol creatinine (Horster et al., 2007, Pediatr. Res. 62: 225-230).

Urine may be collected and urinary MMacid concentrations may be determined using a standard gas chromatography/mass spectroscopy (GC/MS) stable-isotope dilution method.

Plasma Propionylcarnitine, Urinary Methylcitrate, Erythrocyte OLCFAs, Urinary Urea:MMacid Ratio:

Plasma propionylcamitine and urinary methylcitrate are MMacid metabolites and may be used to indicate the effectiveness of a Compound to increase the activity of the relevant enzyme or factor (e.g., MCM, cblA, or cblB). It has been suggested that plasma propionylcamitine may be a more useful measurement than urinary MMacid in the presence of renal insufficiency (Horster et al., 2007, Pediatr. Res. 62: 225-230). Urinary methylcitrate has been found to be elevated in the setting of elevated MMacid levels (Fowler et al., 2008, J. Inherit. Metab. Dis. 31: 350-360). Blood and urine may be collected and standard techniques may be used to determine plasma propionylcamitine levels and urinary methylcitrate levels, respectively.

OLCFAs are measured in erythrocyte membrane lipids and may be used to indicate the effectiveness of a Compound to increase the activity of the relevant enzyme or factor (e.g., MCM, cblA or cblB). Erythrocyte OLCFAs values reflect both the severity of the disease and the quality of the dietary control in MMA (Merinero et al., 2008, J. Inherit. Metab. Dis. 31: 55-66). This parameter is indicative of the propionyl-CoA load of the cells and of long-term metabolic control in organic acidemias (Merinero et al., 2008, J. Inherit. Metab. Dis. 31: 55-66; Sperl et al., 2000, Eur. J. Pediatr. 159: 54-88). A standard method for determination of erythrocyte OLCFAs concentrations is available.

An increase in urinary urea:MMacid ratio following administration of a Compound compared to baseline may indicate an increase in activity of the deficient enzyme or factor (MCM, cblA, or cblB). Protein catabolism leads to production of both urea and MMacid. The values of these catabolic products may fluctuate with dietary protein intake. If the source of MMacid is predominantly natural protein, in patients with no enzyme activity (e.g., mut⁰ patients) the ratio of urinary urea:MMacid is approximately 3.5. If there is residual enzyme activity (e.g., administration of vitamin B12 to cobalamin-sensitive patients) the ratio is generally >5. Patients receiving amino acid supplements will produce more urea than MMacid and will have a urea:MMA ratio>5. However, even in this category of patients the urea:MMA ratio will increase if the activity of the deficient enzyme or factor increases, (Valayannopoulos et al., Annual Symposium of the Society for the Study of Inborn Errors of Metabolism, Amsterdam, The Netherlands, September 2004).

Cellular Enzyme Activity:

The ¹⁴C-propionate incorporation assay is applicable to all patients with MMA, irrespective of enzymatic defect, and measures the overall conversion of propionate to succinate, based on the incorporation of label from 4C—propionate into cell proteins. In mut patients, MCM activity is measured using a radioactive substrate in the presence and absence of Ado-Cbl; and in cblB patients, cobalamin adenosyltransferase activity is measured by monitoring the conversion of OH-[⁵⁷Co]cobalamin to Ado-Cbl (Fowler et al., 2008, J. Inherit. Metab. Dis. 31: 350-360).

Pretreatment cultured fibroblasts for each patient (from an existing cell line or, if that is not available, from a skin biopsy) will be grown in media containing a Compound over a range of concentrations, to assess for changes in propionate incorporation, for all patients, and MCM and cobalamin adenosyltransferase enzyme activity, for mut and cblB patients, respectively.

Lymphocytes may be isolated from blood samples to assess enzyme activity in mut and cblB patients for a change in enzyme activity following Compound administration.

Developmental Delay/Cognition and Dystonia:

Developmental delay and cognitive deficits are among the common neurologic complications of MMA; similarly, dystonia is often observed in patients with the disease (Shevell et al., 1993, Am. J. Med. Genetics 45: 619-624; Nicolaides et al., 1998, Arch. Dis. Child. 78: 508-512). Establishing the feasibility of administering cognitive and dystonia testing in this patient population, characterizing the nature of any dysfunction, and determining the reproducibility of these outcome measures may be useful in assessing the effectiveness of a Compound. Baseline evaluations and evaluations following Compound administration may be performed using standard, age-appropriate developmental/cognitive tests. Examples of the types of tests that may be used include:
Bayley Scale of Infant Development for patients with baseline ages from 2 to <2.5 years
Wechsler Preschool and Primary Scale of Intelligence (WIPPSI) for patients with baseline ages from ≧2.5 years to <6 years
Wechsler Intelligence Scale for Children (WISC) for patients with baseline ages from 6 years to <17 years
Wechsler Adult Intelligence Scale (W AIS) for patients with baseline ages of ≧17 years

The Burke-Fahn-Marsden Movement (BFMM) scale, which is a validated scale for rating dystonia, may be used to measure dystonia (Burke et al., 1985, Neurology 35: 73-77; Karp et al., 1999, Movement Disorders 14: 652-657; Vidailhet et al., 2005, New Eng. J. Med. 352: 459-466; Walker et al., 2000, Movement Disorders 15: 1242-1247).

Metabolic Decompensation Episodes:

Episodes of metabolic decompensation, characterized by vomiting, hypotonia, and alteration of consciousness associated with metabolic acidosis and hyperammonemia, occur in patients with MMA and may be useful in evaluating the effectiveness of a Compound on the treatment of MMA (Horster et al., 2004, Pediatr. Nephrol. 19: 1071-1074; Touati et al., 2006, J. Inherit. Metab. Dis. 29: 288-298). Metabolic decompensation episodes that occur may be documented, with specific assessment of their timing, duration, associated infection or illness, requirement for hospitalization, length of hospitalization, duration of intravenous fluid administration, severity of laboratory abnormalities (e.g., degree of acidosis, lactic acid levels, ammonia levels), and change in level of consciousness.

Safety:

Adverse medical events that may be encountered in patients receiving the Compound may be monitored. For consistency of interpretation, adverse events may be coded using the standard Medical Dictionary for Regulatory Activities (MedDRA), and the severity of these events may be graded using the well-defined Common Terminology Criteria for Adverse Events (CTCAE). Standard definitions for seriousness may be applied.

### Subject Selection

The following eligibility criteria may be used to select subjects for whom treatment with a Compound is considered appropriate.

Subjects should meet the following conditions to be eligible for the treatment protocol:
1. Phenotypic evidence of MMA based on the presence of characteristic clinical symptoms or signs and an elevated plasma MMacid level, such as, e.g., specified in Table 4, infra;
2. Documentation of the presence of a mutation (e.g., nonsense mutation) that results in a premature stop codon in RNA encoded by at least one allele of the MUT or MMAA (cblA) or MMAB (cblB) gene; and
3. In the judgment of the physician, use of a Compound offers acceptable benefit:risk when considering current MMA disease status, medical condition, and the potential benefits of and risks.

TABLE 4

| | | Proposed Eligibility |
|---|---|---|
| System | Parameter | Values |
| Metabolic | Plasma MMacid | >0.27 μmol/L |
| | Serum total bilirubin | ≦1.5 × ULN |
| | Serum GGT | ≦2.5 × ULN |
| | Serum ALT | ≦2.5 × ULN |
| | Serum AST | ≦2.5 × ULN |
| Hepatic | Alkaline phosphatase | ≦2.5 × ULN |
| | Plasma ACTH | ≦ULN |
| | Serum cystatin C | ≦2.0 mg/L |
| Adrenal | GFR | ≦30 mL/min/1.73 m² |
| Renal | Urine blood | ≦1+ |

ACTH = adenocoricotropic hormone,
ALT = alanine aminotransferase,
AST = aspirate aminotransferase,
GFR = glomerular filtration rate,
GGT = gamma glutamyl transferase,
ULN = upper limit of normal

### Compound Administration

A Compound may be orally administered 3 times per day at approximately the same times each day. Ideally doses should be taken at in the morning (e.g., 7:00 am+/−1 hour), at midday (e.g., 1:00 pm+/−1 hour) and in the evening (e.g., 7:00 pm+/−1 hour). The midday dose of the Compound is ideally approximately 6 hours (+/−1 hour) after the morning dose of the Compound, the evening dose of the Compound is ideally approximately 6 hours (+/−1 hour) after the midday dose of the Compound, and the morning dose of the Compound is ideally approximately 12 hours (+/−1 hour) after the evening dose of the Compound. If convenient for the subject, a Compound may be taken during or within approximately 30 minutes after a meal. Subjects may continue receiving repeated 4-week cycles of a Compound indefinitely or until termination. Compound administration may be terminated because of, e.g., disease progression or a dose-limiting toxicity.

Table 5 below provides information on proposed actions to be taken in the event that abnormalities are noted in specified laboratory parameters. Reference should be made to the CTCAE, Version 3.0 (see the website ctep.cancer.gov/forms/CTCAEv3.pdf) for grading severity of adverse events and laboratory abnormalities. Generally, life-threatening (Grade 4) or severe (Grade 3) adverse events or laboratory abnormalities should be considered clinically significant. In addition, clinical evaluations for potential hepatic, adrenal and renal toxicities may be monitored following administration of a Compound.

TABLE 5
Safety Monitoring Parameters and Actions To Be Taken

| | Stop Compound, | | Continue Compound, |
|---|---|---|---|
| Organ System and | Confirm^{a} Abnormal Value, | Confirm^{a} Abnormal Value, then Stop | Confirm^{a} Abnormal Value |
| Laboratory Parameter | and Start Evaluation | Compound and Start Evaluation | and Start Evaluation |
| Hepatic | | | |
| Serum total bilirubin | ≧Grade 3 (≧3.0 × ULN) | Grade 2 (>1.5-3.0 × ULN) | Grade 1 (>ULN-1.5 × |
| | | | ULN)^{b} |
| Serum ALT | ≧Grade 3 (≧5.0 × ULN) | Grade 2 (>2.5-5.0 × ULN) | Grade 1 (>ULN-2.5 × |
| | | | ULN)^{b} |
| Serum AST | ≧Grade 3 (≧5.0 × ULN) | Grade 2 (>2.5-5.0 × ULN) | Grade 1 (>ULN-2.5 × |
| | | | ULN)^{b} |
| Serum GGT | ≧Grade 3 (≧5.0 × ULN) | Grade 2 (>2.5-5.0 × ULN) | Grade 1 (>ULN-2.5 × |
| | | | ULN)^{b} |
| Alkaline phosphatase | ≧Grade 3 (≧5.0 × ULN) | Grade 2 (>2.5-5.0 × ULN) | Grade 1 (>ULN-2.5 × |
| | | | ULN)^{b} |
| Adrenal | | | |
| Plasma ACTH | — | >ULN (and plasma cortisol <LLN) | >ULN (and cortisol WNL) |
| Renal | | | |
| Serum creatinine or | >2.0 × ULN | >1.5-2.0 × ULN | >ULN-1.5 × ULN |
| BUN (if normal at | | | |
| baseline) | | | |
| Serum creatinine or | >2.5 × ULN | >2.0-2.5 × ULN | >1.5-2.0 × ULN |
| BUN (if >ULN-1.5 × | | | |
| ULN at baseline) | | | |
| Serum creatinine or | >3.0 × ULN | >2.5-3.0 × ULN | >2.0-2.5 × ULN |
| BUN (if >1.5-2.0 × | | | |
| ULN at baseline) | | | |
| Serum creatinine or | >4.0 × ULN | >3.5-4.0 × ULN | >3.0-3.5 × ULN |
| BUN (if >2.0-3.0 × | | | |
| ULN at baseline) | | | |
| Serum creatinine or | >5.0 × ULN | >4.5-5.0 × ULN | >4.0-4.5 × ULN |
| BUN(if >3.0 × ULN at | | | |
| baseline) | | | |
| Cystatin C (if WNL | >2.00 mg/L | >1.33-2.00 mg/L | >0.95-1.32 mg/L |
| at baseline) | | | |
| Cystatin C (if >ULN at | — | >2.60 mg/L | >2.0-2.59 mg/L |
| baseline) | | | |

All laboratory abnormalities should be confirmed with a repeat test within 72 hours after the initial abnormal observation.
This criterion applies if baseline value was WNL. If Grade 1 at baseline, refer to preceding 2 columns.
Abbreviations:
ACTH = adrenocorticotropic hormone,
ALT = alanine aminotransferase,
AST = aspartate aminotransferase,
BUN = blood urea nitrogen,
GGT = gamma glutamyl transferase,
LLN = lower limit of normal,
ULN = upper limit of normal for age,
WNL = within normal limits.

A physician should consider whether to continue the course of administration of a Compound to a patient, adjust the dosage of the Compound administered to a patient (e.g., reduce the daily administration of dosages of 20 mg/kg, 20 mg/kg, and 40 mg/kg to 10 mg/kg, 10 mg/k and 20 mg/kg), or discontinue administration of the Compound to a patient if any adverse events or laboratory abnormalities are experienced by a patient.

### Schedule of Events and Procedures:

Medical History:

A medical history should be obtained that focuses on details relating to a subject's MMA and medication usage prior to initial administration of a Compound.

Dietary Information:

dietary information should be obtained prior to initial administration of a Compound, and at other times as clinical indicated. Dietary information that may be obtained includes current type of diet, use of gastric tube feedings, daily protein intake, and use of supplemental amino acids (if any).

MUT/MMAA/MMAB Gene Sequencing:

A verification blood sample for MUT (MCM)/MMAA (cblA)/MMAB (cblB) gene sequencing should be obtained and preserved for confirmatory sequence analysis. A subject who has written documentation of a mutation (e.g., nonsense mutation) that results in a premature stop codon in RNA encoded by an allele of the MUT, MMAA or MMAB gene as the cause for MMA need not wait for confirmatory results to start Compound administration as long as the confirmatory genotyping blood sample has been drawn.

Hepatitis Screen:

An assessment for hepatitis B surface antigen and hepatitis C antibody may be performed prior to initial administration of a Compound, and at other times as clinically indicated.

Plasma Amino Acids:

A plasma sample for amino acids may be to characterize a subject's baseline metabolic status prior to initial administration of a Compound, and at other times as clinically indicated.

Physical Examination:

A physical examination (including evaluation of eyes, ears, nose, mouth, throat, thyroid, lungs, heart, abdomen, extremities, muscular system, skin, lymph nodes, and complete neurologic examination, with special attention to motor and cerebellar aspects) may be conducted prior to initial administration of a Compound, and at other times as clinically indicated.

Dystonia Rating:

The Burke-Fahn-Marsden rating scale for dystonia may be conducted on subjects prior to initial administration of a Compound, and at other times as clinically indicated.

Developmental/Cognitive Testing:

Age-appropriate developmental/cognitive testing may be conducted prior to initial administration of a Compound, and at other times as clinically indicated. For countries in which these tests are available, patients may be administered the following tests, at the appropriate age:
Bayley Scale of Infant Development for patients with baseline ages from 2 to <3 years
WIPPSI for patients with baseline ages from ≧3 years to <6.5 years
WISC for patients with baseline ages from 6.5 years to <17 years
WAIS for patients with baseline ages of ≧17 years
   If any of these specific tests are not available in a particular country, then an equivalent test may be substituted.

Vital Signs:

Vital signs (pulse and blood pressure) may be monitored prior to initial administration of a Compound, and at other times as clinically indicated.

Height and Body Weight:

Height (in cm) can be measured once prior to initial administration of a Compound. Body weight may also be assessed.

Adrenal Laboratory Assessment:

A plasma sample for ACTH and cortisol may be measured prior to administration of a Compound, and at other times as clinically indicated.

Hematology Laboratory Assessment:

Hematology laboratory assessments may include, but are not limited to, white blood cell count with differential, hemoglobin, hematocrit, other red cell parameters, and platelet count. These parameters may be monitored prior to initial administration of a Compound, and during the treatment protocol as necessary.

Biochemistry Laboratory Assessment:

Biochemistry laboratory assessments may include sodium, potassium, chloride, bicarbonate, blood urea nitrogen, creatinine, calcium, phosphorus, uric acid, glucose, total protein, albumin, globulin, albumin:globulin ratio, bilirubin (direct and indirect), aspartate aminotransferase, alanine aminotransferase, gamma glutamyl transferase, alkaline phosphatase, lactate dehydrogenase, total cholesterol, triglycerides, low-density lipoprotein, and high-density lipoprotein. These parameters can be monitored prior to, and/or at various times during, the treatment protocol. To the extent possible, all samples for biochemistry parameter analysis should be taken after an overnight fast.

Cystatin Assessment:

Serum cystatin may be measured monitored prior to, and/or at various times during, the treatment protocol.

Urinalysis:

Urinalyses may include dipstick analysis for pH, specific gravity, glucose, ketones, blood, protein, urobilinogen, bilirubin, nitrate, and leukocytes. These parameters can be monitored prior to initiation of the treatment protocol and/or at various times during the treatment protocol.

Urine Collection for Protein, Creatinine, Osmolality and Renal Biomarkers:

Urine samples may be obtained to measure urine protein, creatinine, osrnolality, and renal biomarkers (such as neutrophil gelatinase-associated lipocalin (N GAL), N acetyl-b-D-glucosaminidase (NAG), retinol binding protein, kidney injury molecule-1 (KIM-1), a microglobulin, (3 microglobulin, microalbumin). These samples may be collected prior to initiation of the treatment protocol and/or at various times during the treatment protocol.

Glomerular Filtration Rate:

GFR may be measured using the infusion method available (such as 51Cr-ethylenediaminetetraacetic acid [EDTA], iohexyl, or inulin) prior to initiation of the treatment protocol and/or at various times during the treatment protocol.

Renal Ultrasound:

An ultrasound to examine the kidneys and collecting system may be performed prior to initiation of the treatment protocol and/or at various times during the treatment protocol.

12-Lead Electrocardiogram:

A 12-lead ECG can be obtained prior to initiation of the treatment protocol and/or at various times during the treatment protocol.

Blood for a Compound's Plasma Concentrations:

Blood samples for a Compound's plasma concentration assessments can be collected immediately at, e.g., about 1, 2, 3 and 4 hours after administration of the morning dose, at, e.g., about 1, 2, 3 and 4 hours after administration of the midday dose, and immediately pre-dose and at, e.g., about 1, 2, 3 and 4 hours after administration of the evening dose. Such collections can be done at various time points during the treatment protocol

Analyses of a Compound's plasma concentrations can be performed using a validated HPLC-MS/MS method. Plasma samples collected for analysis can be preserved for future metabolite analysis, as appropriate.

Plasma MMacid:

Plasma samples for MMacid levels may be assessed prior to initiation of the treatment protocol and/or at various times during the treatment protocol.

Urinary MMacid:

Urinary MMacid levels may be assessed prior to initiation of the treatment protocol and/or at various times during the treatment protocol.

Urinary Urea:

Urine samples for assessing urea can be collected prior to initiation of the treatment protocol and/or at various times during the treatment protocol.

Urinary Methylcitrate:

Urine samples for assessing urinary methylcitrate can be collected prior to initiation of the treatment protocol and/or at various times during the treatment protocol.

Erythrocyte Odd Long-Chain Fatty Acid Levels:

Blood may be collected and erythrocyte OLCFA levels may be assessed prior to initiation of the treatment protocol and/or at various times during the treatment protocol.

Assessment of Metabolic Decompensation Episodes:

During the treatment protocol, episodes of metabolic decompensation may be recorded, with specific assessment of timing, associated infection or illness, duration, requirement for hospitalization, length of hospitalization, duration of intravenous fluid administration, severity of laboratory abnormalities (including acidosis, lactic acid levels, and ammonia levels), and change in level of consciousness.

Skin Biopsy:

Skin biopsy may be performed prior to initiation of the treatment protocol to obtain and culture fibroblasts if a fibroblast cell line not already available for the patient. If patient has an existing fibroblast cell line but it is found not to be viable for enzyme analysis, then skin biopsy should be performed when this becomes known at any time during the study.

Skin biopsy may be performed under local anesthesia, as considered appropriate for a specific subject or study site. Cultured fibroblasts for each patient (from existing cell lines or from a skin biopsy) may be grown in media containing Compound over a range of concentrations, to assess for changes in propionate incorporation, for all patients, and for MCM and cobalamin adenosyltransferase enzyme activity, for mut and cblB patients, respectively.

Cellular Enzyme Activity:

Lymphocytes isolated from blood samples may be assayed for enzyme activity, in mut and cblB patients, prior to initiation of the treatment protocol and at various times during the treatment protocol.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. A method for treating methylmalonic academia (MMA), comprising administering to a human having a mutation in at least one allele of the MUT, MMAA (cblA) or MMAB (cblB) gene that results in a premature stop codon in RNA encoded by an allele of the MUT, MMAA or MMAB gene an effective amount of a compound having formula I: 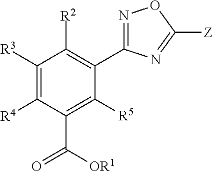
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl;
R¹ is hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, —(CH₂CH₂O)ₙR⁶ or a biohydrolyzable group;
R², R³, R⁴, R⁵ and R⁶ are independently hydrogen, hydroxyl, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen, CF₃, OCF₃, OCHF₂, CN, COOH, COOR⁷, SO₂R⁷, NO₂, NH₂, or N(R⁷)₂;
each occurrence of R⁷ is independently hydrogen, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen or CF₃; and
n is an integer from 1 to 7.

2. The method of claim 1, wherein the compound has formula II: 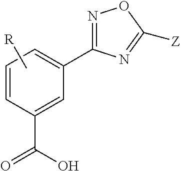
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, or substituted or unsubstituted arylalkyl; and R is hydrogen or halogen.

3. The method of claim 2, wherein the compound has formula III: 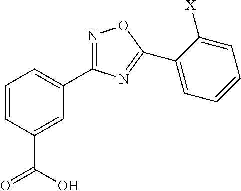
or a pharmaceutically acceptable salt, hydrate, solvate or stereoisomer thereof wherein:
X is halogen, substituted alkyl or substituted or unsubstituted alkoxy.

4. The method of claim 3, wherein the compound having formula III is: 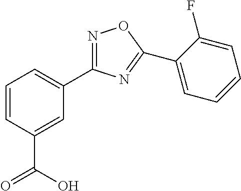 [SMILES: O=C(O)c1cccc(-c2noc(-c3ccccc3F)n2)c1]
or a pharmaceutically acceptable salt, hydrate, solvate or stereoisomer thereof.

5. The method of claim 1, wherein the mutation is a nonsense mutation.

6. A method for treating MMA, comprising administering to a human having a mutation in at least one allele of the MUT gene that results in a premature stop codon in RNA encoded by an allele of the MUT gene an effective amount of a compound having formula I: 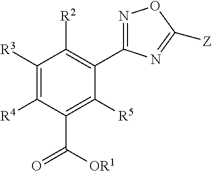
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl;
R¹ is hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, —(CH₂CH₂O)ₙR⁶ or a biohydrolyzable group;
R², R³, R⁴, R⁵ and R⁶ are independently hydrogen, hydroxyl, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen, CF₃, OCF₃, OCHF₂, CN, COOH, COOR⁷, SO₂R⁷, NO₂, NH₂, or N(R⁷)₂;
each occurrence of R⁷ is independently hydrogen, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen or CF₃; and
n is an integer from 1 to 7,
wherein the mutation is the result of one or more nucleotide changes in an exon selected from Exon 5: c.1025 C>A, Exon 2: c.19 C>T, or c.52C>T, Exon 6: c.1237 C>T, c.682 C>T, c.1207 C>T, or c.1240 G>T, Exon 7: c.1423 C>T, or c.1399 C>T, and Exon 8: c.1531 C>T, c.454 C>T, c.397 C>T, c.433 C>T or c.358 C>T.

7. The method of claim 6, wherein the compound has formula II: 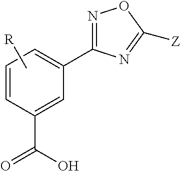
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, or substituted or unsubstituted arylalkyl; and R is hydrogen or halogen.

8. The method of claim 7, wherein the compound has formula III: 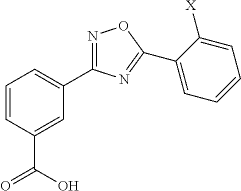
or a pharmaceutically acceptable salt, hydrate, solvate or stereoisomer thereof wherein:
X is halogen, substituted alkyl or substituted or unsubstituted alkoxy.

9. The method of claim 8, wherein the compound having formula III is: 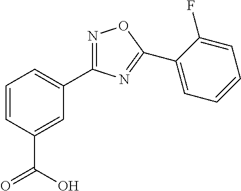 [SMILES: O=C(O)c1cccc(-c2noc(-c3ccccc3F)n2)c1]
or a pharmaceutically acceptable salt, hydrate, solvate or stereoisomer thereof.

10. The method of claim 6, wherein the mutation is a nonsense mutation.

11. A method for treating MMA, comprising administering to a human having a mutation in at least one allele of the MMAA gene that results in a premature stop codon in RNA encoded by an allele of the MMAA gene an effective amount of a compound having formula I: 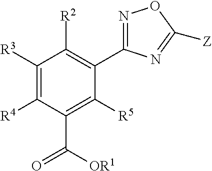
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl;
R¹ is hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, —(CH₂CH₂O)ₙR⁶ or a biohydrolyzable group;
R², R³, R⁴, R⁵ and R⁶ are independently hydrogen, hydroxyl, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen, CF₃, OCF₃, OCHF₂, CN, COOH, COOR⁷, SO₂R⁷, NO₂, NH₂, or N(R⁷)₂;
each occurrence of R⁷ is independently hydrogen, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen or CF₃; and
n is an integer from 1 to 7,
wherein the mutation is the result of one or more nucleotide changes in an exon selected from Exon 4: c.812_{—}813 dupAG, Exon 3: c.594 dupT, or Exon 2: c.450dupG, c.885 C>T, or c.433 C>T.

12. The method of claim 11, wherein the compound has formula II: 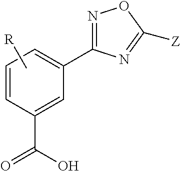
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, or substituted or unsubstituted arylalkyl; and R is hydrogen or halogen.

13. The method of claim 12, wherein the compound has formula III: 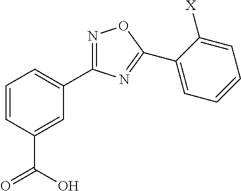
or a pharmaceutically acceptable salt, hydrate, solvate or stereoisomer thereof wherein:
X is halogen, substituted alkyl or substituted or unsubstituted alkoxy.

14. The method of claim 13, wherein the compound having formula III is: 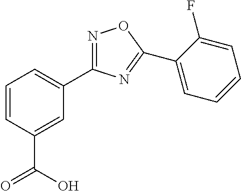 [SMILES: O=C(O)c1cccc(-c2noc(-c3ccccc3F)n2)c1]
or a pharmaceutically acceptable salt, hydrate, solvate or stereoisomer thereof.

15. The method of claim 11, wherein the mutation is a nonsense mutation.

16. A method for treating MMA, comprising administering to a human having a mutation in at least one allele of the MMAB gene that results in a premature stop codon in RNA encoded by an allele of the MMAB gene an effective amount of a compound having formula I: 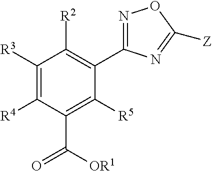
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, substituted or unsubstituted arylalkyl;
R¹ is hydrogen, substituted or unsubstituted alkyl, substituted or unsubstituted cycloalkyl, substituted or unsubstituted heterocycloalkyl, substituted or unsubstituted aryl, substituted or unsubstituted heteroaryl, —(CH₂CH₂O)ₙR⁶ or a biohydrolyzable group;
R², R³, R⁴, R⁵ and R⁶ are independently hydrogen, hydroxyl, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen, CF₃, OCF₃, OCHF₂, CN, COOH, COOR⁷, SO₂R⁷, NO₂, NH₂, or N(R⁷)₂;
each occurrence of R⁷ is independently hydrogen, substituted or unsubstituted alkyl, substituted or unsubstituted alkenyl, substituted or unsubstituted alkynyl; substituted or unsubstituted cycloalkyl, substituted or unsubstituted heterocycloalkyl, substituted or unsubstituted aryl, substituted or unsubstituted heteroaryl, alkoxy, aryloxy, heteroaryloxy, halogen or CF₃; and
n is an integer from 1 to 7,
wherein the mutation is the result of one or more nucleotide changes in an intron selected from Intron 3: c.291-1G>A.

17. The method of claim 16, wherein the compound has formula II: 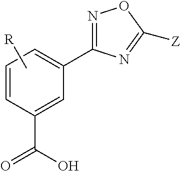
or a pharmaceutically acceptable salt, hydrate, solvate or stereoisomer thereof wherein:
Z is substituted or unsubstituted aryl, substituted or unsubstituted heteroaryl, substituted or unsubstituted cycloalkyl, substituted or unsubstituted alkyl, substituted or unsubstituted alkenyl, substituted or unsubstituted heterocycle, or substituted or unsubstituted arylalkyl; and R is hydrogen or halogen.

18. The method of claim 17, wherein the compound has formula III: 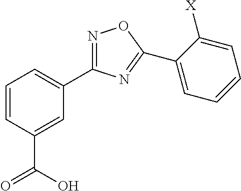
or a pharmaceutically acceptable salt, hydrate, solvate or stereoisomer thereof wherein:
X is halogen, substituted alkyl or substituted or unsubstituted alkoxy.

19. The method of claim 18, wherein the compound having formula III is: 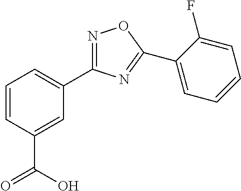 [SMILES: O=C(O)c1cccc(-c2noc(-c3ccccc3F)n2)c1]
or a pharmaceutically acceptable salt, hydrate, solvate or stereoisomer thereof.

20. The method of claim 16, wherein the mutation is a nonsense mutation.

21. The method of claim 1, wherein the method further comprises administering the compound of claim 1 or a pharmaceutically acceptable salt, hydrate, solvate or stereoisomer thereof in a combination therapy with one or more agents selected from a carnitine supplement, a cobalamin supplement or an antibiotic.

22. The method of claim 21, wherein the carnmitine supplement is L-camitine and the antibiotic is metronidazole.
